SEQUENCE LISTING (22 sequences)

SEQ ID NO: 1 - DNA, 4661 bp, Homo sapiens
attttgcatgccaccttaatctttttttttttttttttaaatcgaggtttcagtctcattctatttcccaggctggagttcaatagcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgttaatcttgaggtacataaacctggctcctaaaggctaaaggctaaatatttgttggagaaggggcattggattttgcatgaggatgattctgacctgggagggcaggtcagcaggcatctctgttgcacagatagagtgtacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagctacagggcctggtgcatccagggtgatctagtaattgcagaacagcaagtactagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctccatattgcaacagtcctcactcccacaccaggtccccgctccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccacctcctgaggccctcaatcctatcacagtctactgacttttcccattcagctgtgagtgcccaaccctatcccagagaccttgatgcttggcctcccaatcttgccctaggatacccagatgccaaccagacacctccttcttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtgggrtacagagatgaaagaggggtgggwwccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctsttatacgtggtttattctggtttgttacattgacaggagacacactgaaatcagcaaaggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagttcttattgggggtctgtaggataggcatggggtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcatctcccaggagttattcaagggtgagccctgtacttggaacgttcaggcttt

SEQ ID NO: 2 - DNA, 4661 bp, Homo sapiens; identical to SEQ ID NO: 1

SEQ ID NO: 3 - DNA, 3846 bp, Homo sapiens
attttgcatgccaccttaatctttttttttttttttttaaatcgaggtttcagtctcattctatttcccaggctggagttcaatagcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgttaatcttgaggtacataaacctggctcctaaaggctaaaggctaaatatttgttggagaaggggcattggattttgcatgaggatgattctgacctgggagggcaggtcagcaggcatctctgttgcacagatagagtgtacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagctacagggcctggtgcatccagggtgatctagtaattgcagaacagcaagtactagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctccatattgcaacagtcctcactcccacaccaggtccccgctccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccacctcctgaggccctcaatcctatcacagtctactgacttttcccattcagctgtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtgggrtacagagatgaaagaggggtgggwwccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctsttatacgtggtttattctggtttgttacattgacaggagacacactgaaatcagcaaaggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagttcttattgggggtctgtaggataggcatggggtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcatctcccaggagttattcaagggtgagccctgtacttggaacgttcaggcttt

SEQ ID NO: 4 - DNA, 1709 bp, Homo sapiens
attttgcatgccaccttaatctttttttttttttttttaaatcgaggtttcagtctcattctatttcccaggctggagttcaatagcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgttaatcttgaggtacataaacctggctcctaaaggctaaaggctaaatatttgttggagaaggggcattggattttgcatgaggatgattctgacctgggagggcaggtcagcaggcatctctgttgcacagatagagtgtacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagctacagggcctggtgcatccagggtgatctagtaattgcagaacagcaagtactagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaactccctgctcccagctgcttaa

SEQ ID NO: 5 - DNA, 3423 bp, Homo sapiens
attttgcatgccaccttaatctttttttttttttttttaaatcgaggtttcagtctcattctatttcccaggctggagttcaatagcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgttaatcttgaggtacataaacctggctcctaaaggctaaaggctaaatatttgttggagaaggggcattggattttgcatgaggatgattctgacctgggagggcaggtcagcaggcatctctgttgcacagatagagtgtacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagctacagggcctggtgcatccagggtgatctagtaattgcagaacagcaagtactagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtgggrtacagagatgaaagaggggtgggwwccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctsttatacgtggtttattctggtttgttacattgacaggagacacactgaaatcagcaaaggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagttcttattgggggtctgtaggataggcatggggtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcatctcccaggagttattcaagggtgagccctgtacttggaacgttcaggcttt

SEQ ID NO: 6 - DNA, 1261 bp, Homo sapiens
gggcggggtcctggagaatgaaggctttatagggctcctcagggaggccccccagccccaaactgcaccacctggccgtggacacctgtgtcagcatgtgggacctggttctctccatcgccttgtctgtggggtgcactggtgagattggggggataaaggaaggggggcgggttctgactcttatgctgaagcccttttcctcccacccagtgccccagcctcgtcccttcagcccacagttcagcccagacaatgtgcccctgactcttccacattgcaatagtcctcatgcccacactaggtccccgctccctcccacttacctcagacctttctctccattgcccagccaaatccctgctcccagctgctttactaaagagcaagttcctaggcatctctgtgtttctctttatggggttcaaaacctttcaaggacctctctccatgccactggttccttggaccctatcactgggctgcctcctgagcccctcagtcctaccacagtctactgacttttcccattcagctgtgagcattcaaccctgtcccctggaccttgacacctggctccccaaccctgtcccaggaaacccagattccaccagacacttccttcttcccccccgaggctatctggcctgagacaacaaatgctgcctcccaccctgagtctggcactgggactttcagaactcctccttccctgactctttgccccagacccgtcattcaatggctagctttttccatgggaagaagaacaacgagcacccccaaccacaacggccagttctctgattccctaaatccgcacccttttcaaaacctcaaaaacaaaacaaaacaaaacaaagcaagaaacaactcaggcaaaacttgttgcttaaccttggacatggtaaaccatccaaaaccttcctctcccagcaactaaacctctccactgggcacttaacctttggtttcttggaacctcttaatctcttagaacccacagctgccaccacatgcccttctcccaatgtaagaccccaaatcactccaaatgacccaacccccaacccatgcctccttcagatatttcccatgtcccctactctgatctctggggtcagctccgttctcgagagcatgaagcctcccgacctggtccagccaccaacccgctaacgcagggaatagctacagaattgccagccctcccaggaccccttgcttgtgtcctggactcccagtcctggtcctctgcccc

SEQ ID NO: 7 - protein, 104 aa, Homo sapiens
MWVPVVFLTLSVTWIGERGHGWGDAGEGASPDCQAEALSPPTQHPSPDRELGSFLSLPAPLQAHTPSPSILQQSSLPHQVPAPSHLPQNFLPIAQPAPCSQLLY

SEQ ID NO: 8 - protein, 218 aa, Homo sapiens [sequence omitted]

SEQ ID NO: 9 - protein, 218 aa, Homo sapiens [sequence omitted]

SEQ ID NO: 10 - protein, 183 aa, Homo sapiens
MKNRGSYPPPVSVSSWACLLCLCPLDEVSMSYRAWCIQGDLVIAEQQVLALPPLPQLWVWEGVVQPPAAWGGPWSASGCQQGRGGVLGNEGFIGLLGEAPQPQAYHLHPESCVTMWVPVVFLTLSVTWIGERGHGWGDAGEGASPDCQAEALSPPTQHPSPDRELGSFLSLPAPLQLPAPSCL

SEQ ID NO: 11 - protein, 375 aa, Homo sapiens
MKNRGSYPPPVSVSSWACLLCLCPLDEVSMSYRAWCIQGDLVIAEQQVLALPPLPQLWVWEGVVQPPAAWGGPWSASGCQQGRGGVLGNEGFIGLLGEAPQPQAYHLHPESCVTMWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 12 - protein, 141 aa, Homo sapiens [sequence omitted]

SEQ ID NO: 13 - DNA, 10574 bp, Homo sapiens
aagcttctagttttcttttcccggtgacatcgtggaaagcactagcatctctaagcaatgatctgtgacaatattcacagtgtaatgccatccagggaactcaactgagccttgatgtccagagatttttgtgtttttttctgagactgagtctcgctctgtgccaggctggagtgcagtggtgcaaccttggctcactgcaagctccgcctcctgggttcacgccattctcctgcctcagcctcctgagtagctgggactacaggcacccgccaccacgcctggctaatttttttgtatttttagtagagatggggtttcactgtgttagccaggatggtctcagtctcctgacctcgtgatctgcccaccttggcctcccaaagtgctgggatgacaggcgtgagccaccgcgcctggccgatatccagagattttttggggggctccatcacacagacatgttgactgtcttcatggttgacttttagtatccagcccctctagaaatctagctgatatagtgtggctcaaaaccttcagcacaaatcacaccgttagactatctggtgtggcccaaaccttcaggtgaacaaagggactctaatctggcaggatattccaaagcattagagatgacctcttgcaaagaaaaagaaatggaaaagaaaaagaaagaaaggaaaaaaaaaaaaaaaaagagatgacctctcaggctctgaggggaaacgcctgaggtctttgagcaaggtcagtcctctgttgcacagtctccctcacagggtcattgtgacgatcaaatgtggtcacgtgtatgaggcaccagcacatgcctggctctggggagtgccgtgtaagtgtatgcttgcactgctgaatgcttgggatgtgtcagggattatcttcagcacttacagatgctcatctcatcctcacagcatcactatgggatgggtattactggcctcatttgatggagaaagtggctgtggctcagaaaggggggaccactagaccagggacactctggatgctggggactccagagaccatgaccactcaccaactgcagagaaattaattgtggcctgatgtccctgtcctggagagggtggaggtggaccttcactaacctcctaccttgaccctctcttttagggctctttctgacctccaccatggtactaggaccccattgtattctgtaccctcttgactctatgacccccactgcccactgcatccagctgggtcccctcctatctctattcccagctggccagtgcagtctcagtgcccacctgtttgtcagtaactctgaaggggctgacattttactgacttgcaaacaaataagctaactttccagagttttgtgaatgctggcagagtccatgagactcctgagtcagaggcaaaggcttttactgctcacagcttagcagacagcatgaggttcatgttcacattagtacaccttgccccccccaaatcttgtagggtgaccagagcagtctaggtggatgctgtgcagaaggggtttgtgccactggtgagaaacctgagattaggaatcctcaatcttatactgggacaacttgcaaacctgctcagcctttgtctctgatgaagatattatcttcatgatcttggattgaaaacagacctactctggaggaacatattgtatcgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggagaattgcctcccaacgctgttcagccagagccttccacccttgtctgcaggacagtctcaacgttccaccattaaatacttcttctatcacatcctgcttctttatgcctaaccaaggttctaggtcccgatcgactgtgtctggcagcactccactgccaaacccagaataaggcagcgctcaggatcccgaaggggcatggctggggatcagaacttctgggtttgagtgaggagtgggtccaccctcttgaatttcaaaggaggaagaggctggatgtgaaggtactgggggagggaaagtgtcagttccgaactcttaggtcaatgagggaggagactggtaaggtcccagctcccgaggtactgatgtgggaatggcctaagaatctcatatcctcaggaagaaggtgctggaatcctgaggggtagagttctgggtatatttgtggcttaaggctctttggcccctgaaggcagaggctggaaccattaggtccagggtttggggtgatagtaatgggatctcttgattcctcaagagtctgaggatcgagggttgcccattcttccatcttgccacctaatccttactccacttgagggtatcaccagcccttctagctccatgaaggtcccctgggcaagcacaatctgagcatgaaagatgccccagaggccttgggtgtcatccactcatcatccagcatcacactctgagggtgtggccagcaccatgacgtcatgttgctgtgactatccctgcagcgtgcctctccagccacctgccaaccgtagagctgcccatcctcctctggtgggagtggcctgcatggtgccaggctgaggcctagtgtcagacagggagcctggaatcatagggatccaggactcaaaagtgctagagaatggccatatgtcaccatccatgaaatctcaagggcttctgggtggagggcacagggacctgaacttatggtttcccaagtctattgctctcccaagtgagtctcccagatacgaggcactgtgccagcatcagccttatctccaccacatcttgtaaaaggactacccagggccctgatgaacaccatggtgtgtacaggagtagggggtggaggcacggactcctgtgaggtcacagccaagggagcatcatcatgggtggggaggaggcaatggacaggcttgagaacggggatgtggttgtatttggttttctttggttagataaagtgctgggtataggattgagagtggagtatgaagaccagttaggatggaggatcagattggagttgggttagataaagtgctgggtataggattgagagtggagtatgaagaccagttaggatggaggatcagattggagttgggttagagatggggtaaaattgtgctccggatgagtttgggattgacactgtggaggtggtttgggatggcatggctttgggatggaaatagatttgttttgatgttggctcagacatccttggggattgaactggggatgaagctgggtttgattttggaggtagaagacgtggaagtagctgtcagatttgacagtggccatgagttttgtttgatggggaatcaaacaatgggggaagacataagggttggcttgttaggttaagttgcgttgggttgatggggtcggggctgtgtataatgcagttggattggtttgtattaaattgggttgggtcaggttttggttgaggatgagttgaggatatgcttggggacaccggatccatgaggttctcactggagtggagacaaacttcctttccaggatgaatccagggaagccttaattcacgtgtaggggaggtcaggccactggctaagtatatccttccactccagctctaagatggtcttaaattgtgattatctatatccacttctgtctccctcactgtgcttggagtttacctgatcactcaactagaaacaggggaagattttatcaaattcttttttttttttttttttttttgagacagagtctcactctgttgcccaggctggagtgcagtggcgcagtctcggctcactgcaacctctgcctcccaggttcaagtgattctcctgcctcagcctcctgagttgctgggattacaggcatgcagcaccatgcccagctaatttttgtatttttagtagagatggggtttcaccaatgtttgccaggctggcctcgaactcctgacctggtgatccacctgcctcagcctcccaaagtgctgggattacaggcgtcagccaccgcgcccagccacttttgtcaaattcttgagacacagctcgggctggatcaagtgagctactctggttttattgaacagctgaaataaccaactttttggaaattgatgaaatcttacggagttaacagtggaggtaccagggctcttaagagttcccgattctcttctgagactacaaattgtgattttgcatgccaccttaatctttttttttttttttttaaatcgaggtttcagtctcattctatttcccaggctggagttcaatagcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgttaatcttgaggtacataaacctggctcctaaaggctaaaggctaaatatttgttggagaaggggcattggattttgcatgaggatgattctgacctgggagggcaggtcagcaggcatctctgttgcacagatagagtgtacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctccatattgcaacagtcctcactcccacaccaggtccccgctccctcccacttaccccagaactttctccccatttgcccagccagctccctgctcccagctgctttactaaaggggaagttcctgggcatctccgtgtttctctttgtggggctcaaaacctccaaggacctctctcaatgccattggttccttggaccgtatcactggtccacctcctgagcccctcaatcctatcacagtctactgacttttccattcagctgtgagtgcccaaccctatcccagagaccttgatgcttggcctcccaatcttgccctaggatacccagatgccaaccagacacctccttcttcctagccaggctatctggctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcccgataaggcacttatccctggttcctagcaccgcttatcccctcagaatccacaacttgtaccaagtttcccttctcccagtccaagaccccaaatcaccacaaaggacccaatccccagactcaagatatggtctggggctgtcttgtgtctcctaccctgatccctgggttcaactctgtcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtccagaggaataacagctgggcattttccccaggataacctctaaggccagccttgggactgggggagagagggaaagttctggttcaggtcacatggggaggcagggttggggctggaccaccctccccatggctgcctgggtctccatctgtgttcctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgagcacacgcatgggatgggcctggggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtcggcgcatgcctgtagtcccagctactcaggaggctgagggaggagaattgcttgaacctgggaggtggaggttgcagtgagccgagacgtgccactgcactccagcctgggtgacagagtgagactccgcctcaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttcagctggagctgggaggggcaattgagggaggaggaaggagaagggggaaggaaaacagggtatgggggaaaggaccctggggagcgaagtggaggatacaaccttgggcctgcaggccaggctacctacccacttggaaacccacgccaaagccgcatctacagctgagccactctgaggcctcccctccccagcggtccccactcagctccaaagtctctctcccttttctctcccacactctatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgtccttttaccctcttcctttttcccttggtttctctcagtttctgtatctgcccttcaccctctcacactgctgtttcccaactcgttgtctgtatttttggcctgaacatgtgtcttccccaaccctgtgtttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctaggtctgagggaggagggccaaggaaccaggtggggtccagcccacaacagtgttttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgaggggaaaggtgagtggggaccttaattctgggctggggtctagaagccaacaagcatctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggcgggagtgagcaaacacctgccgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaagaaggacccctcctgcagggcctcacctgggccacaggaggacactgcttttcctctgaggagtcaggaactgtggatggtgctggacagaagcaggacagggcctggctcaggtgtccagaggctgccgctggcctccctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctg

SEQ ID NO: 14 - protein, 262 aa, Homo sapiens [sequence omitted]

SEQ ID NO: 15 - protein, 18 aa, Homo sapiens
MWDLVLSIALSVGCTGAV

SEQ ID NO: 16 - protein, 18 aa, Homo sapiens
CQAELSPPTQHPSPDREL

SEQ ID NO: 17 - DNA, 1466 bp, Homo sapiens
agccccaagcttaccacctgcacccggagagctgtgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 18 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 19 - DNA, 901 bp, Homo sapiens
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcaaaaaaaaaaaaaaaaaaaaaaaaaaaaa

SEQ ID NO: 20 - protein, 261 aa, Homo sapiens
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 21 - protein, 20 aa, Homo sapiens
PSQIPAPSCFTKEQVPRHLC

SEQ ID NO: 22 - DNA, 781 bp, Homo sapiens
ccaacccagcaccccagcccagacagggagctgggctcttttctgtctctcccagccccactccaagcccatacccccagcccctccatattgcaacagtcctcactcccacaccaggtccccgctccctcccacttaccccagaactttctccccatttgcccagccagctccctgctcccagctgctttactaaaggggaagttcctgggcatctccgtgtttctctttgtggggctcaaaacctccaaggacctctctcaatgccattggttccttggaccgtatcactggtccacctcctgagcccctcaatcctatcacagtctactgacttttccattcagctgtgagtgcccaaccctatcccagagaccttgatgcttggcctcccaatcttgccctaggatacccagatgccaaccagacacctccttcttcctagccaggctatctggctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcccgataaggcacttatccctggttcctagcacc

(19) United States
(12) Patent Application Publication
(10) Pub. No.: US 2003/0207808 A1
(43) Pub. Date: Nov. 6, 2003

(54) Novel nucleic acid and amino acid sequences

(76) Inventors: Kinneret Savitzky, Tel Aviv (IL); Liat Mintz, Ramat Hasharon (IL); Anat David, Givataim (IL); Idit Azar, Rehovot (IL)

Correspondence Address: BIRCH STEWART KOLASCH & BIRCH, PO BOX 747, FALLS CHURCH, VA, 22040-0747, US

(21) Appl. No.: 10/298,965

(22) Filed: Nov. 19, 2002

Related U.S. Application Data
(63) Continuation-in-part of application No. 09/755,100, filed on Jan. 8, 2001.
Application No. 09/755,100 is a continuation-in-part of application No. 09/701,238, now abandoned.
Application No. 09/701,238 is a 371 of international application No. PCT/IL00/00102, filed on Feb. 18, 2000.

(30) Foreign Application Priority Data
Feb. 18, 1999 (IL) 128587
Apr. 14, 1999 (IL) 129439
Aug. 11, 1999 (IL) 131363

(51) Int. Cl. C12Q 1/68; C07H 21/04; C12P 21/02; C12N 5/06; C07K 14/47; A61K 38/17; A61K 48/00; A61K 39/395

(52) U.S. Cl. 514/12; 514/44; 424/155.1; 435/6; 435/320.1; 435/325; 530/350; 530/388.8; 536/23.5; 435/69.3

(57) ABSTRACT

The invention concerns novel nucleic acid sequences, amino acid sequences coded thereby and method of detection using the above. The novel nucleic acid sequences are naturally occurring splice variants of a prostate specific antigen sequence (PSA) or of the KLK-2 gene.

### FIELD OF THE INVENTION

This patent application is a Continuation-In-Part of U.S. patent application Ser. No. 09/755,100 dated Jan. 8, 2001; which is a Continuation-In-Part of U.S. patent application Ser. No. 09/701,238, which is National Phase of PCT/IL00/00102, which claims priority from Israel Applications Nos. 131363, 129439 and 128587, which are all incorporated by reference.

The present invention concerns novel nucleic acid sequences, vectors and host cells containing them, amino acid sequences encoded by said sequences, and antibodies reactive with said amino acid sequences, as well as pharmaceutical compositions comprising any of the above. The present invention further concerns methods for screening for candidate activator or deactivators utilizing said amino acid sequences.

### BACKGROUND OF THE INVENTION

Prostate-specific antigen (PSA) is the most important tumor marker for early detection, staging, and monitoring of men with prostate cancer today. PSA testing has appreciable false-positive and false-negative results, particularly in the 2.5-10 ng/ml range. Measurement of the percentage of non-protein-bound (i.e. free) PSA in serum, which is lower in patients with prostate cancer, have been evaluated as a method for increasing the accuracy of PSA testing.

Thus measurement of PSA in serum, has been postulated as having potential clinical utility for increasing the sensitivity and specificity of PSA testing. Cutoff figures are affected by total PSA levels at prostate value. The prevalence rate of cancer in the screened population, depending on age, race, previous biopsy history etc., also influences the screening cutoffs. It has also been postulated that the percentage of free PSA may also correlate with a potential aggressiveness of early-stage prostate cancer. Thus, the level of free PSA may not only be used in order to diagnose prostate cancer, but also to predict the course of development of this cancer, and the patient's prognosis, and decide on a suitable treatment regime.

Human kallikrein-2 gene (termed herein after: “KLK” which is also known as KLK-2) is transcribed from the same locus as the PSA and is also known to be prostate specific. It has been speculated that both PSA and KLK have common expression control such as common enhancer and/or promoter and both function as serine proteases.

### GLOSSARY

In the following description and claims use will be made, at times, with a variety of terms, and the meaning of such terms as they should be construed in accordance with the invention is as follows:

“Prostate specific antigen (PSA) variant”—the sequence shown in any one of SEQ ID NO: 1 to SEQ ID NO: 6, sequences having at least 70% identity to said sequence and fragments of the above sequences of least 20 b p. long. SEQ ID NO: 1 to ID NO: 5 are nucleic acid sequences which resulted from alternative splicing of the native and known PSA sequence appearing in HSPSAR and HUMPSANTIG (GenBank Acc. X05332 and M24543, respectively, depicted in SEQ ID NO: 17 for nucleotides and SEQ ID NO: 18 for amino acids). It should be emphasized that the PSA variants of the invention are naturally occurring sequences resulting from the alternative splicing of the RNA transcribed from the PSA gene and not merely truncated or mutated forms of the gene. SEQ ID NO: 6 is an alternative splice variant of the human kallikrein-2 gene (KLK-2) appearing in GenBank as KLK2 under Accession Number NM_{—}005551, depicted in SEQ ID NO: 19 for nucleotide sequence and SEQ ID NO: 20 for amino acid sequences.

SEQ ID NO: 1—(PSAL_{—}0): The nucleic acid sequence starting in position 4364 of the HUMPSANTIG up to position 7305, then a different sequence. The coded peptide (SEQ ID NO: 7) starting identically to the original PSA for 16 aa, then a different sequence which is transcribed from the PSA intron between exons 1 and 2.

SEQ ID NO: 2—(PSAL_{—}1):

Nucleic acid sequence identical to SEQ 1.

Peptide (SEQ ID NO: 8)—Starting in a Methionine 114 aa upstream from the original PSA, and has the same 16 aa identity and 3′ end as PSAL_{—}0.

SEQ ID NO: 3—(PSAL_{—}2):

Nucleic acid sequence which starts in same place as PSAL_{—}0 but goes up to position 6336 of the HUMPSANTIG, then continues in a different sequence.

Peptide (SEQ ID NO: 9)—Identical to PSAL_{—}1 peptide.

SEQ ID NO: 4—(PSAL_{—}5):

Nucleic acid sequence which starts in same place as PSAL_{—}0, goes up to position 6069 of HUMPSANTIG and end there (original intron).

Peptide (SEQ ID NO: 10)—Has same starting place as PSAL_{—}1, the same 16 aa identity to PSA, then a different intron region translated.

SEQ ID NO: 5—(PSAL_{—}6):

Nucleic acid sequence starts in the same place as PSAL_{—}0, goes up to position 5913 of HUMPSANTIG, then enter the original PSA exon #2 and continues.

Peptide (SEQ ID NO: 11) has same starting place as PSAL_{—}1, then enters the same identity region and continues as the original PSA until the end.

SEQ ID NO: 6 is a splice variant of the KLK-2 that includes coding region from the original KLK-2 intron between exons 1 and 2. The term of “PSA variant” in the context of the present invention concerns splice variants of the known PSA gene as well as splice variants of the KLK-2 gene, which is also known to code for antigens specific to the prostate. An example of a protein, encoded by SEQ ID NO: 6 is depicted in SEQ ID NO: 12.

“Prostate specific antigen variant product (PSA variant products)—also referred at times as the “PSA variant protein” or “PSA variant polypeptide”—an amino acid sequence coded by said PSA variant nucleic acid sequence. The amino acid sequence may be a peptide, a protein, as well as peptides or proteins having chemically modified amino acids (see below) such as a glycopeptide or glycoprotein. An example of a PSA variant product is shown in any one of SEQ ID NO: 7 to SEQ ID NO: 12, and includes also analogues of said sequences in which one or more amino acids has been added, deleted, substituted (see below) or chemical modified (see below) as well as fragments of this sequence having at least 10 amino acids. The products may be membrane associated or present in a free form in body fluids, for example in the serum.

“Nucleic acid sequence”—a sequence composed of DNA nucleotides, RNA nucleotides or a combination of both types and may includes natural nucleotides, chemically modified nucleotides and synthetic nucleotides.

“Amino acid sequence”—a sequence composed of any one of the 20 naturally appearing amino acids, amino acids which have been chemically modified (see below), or composed of synthetic amino acids.

“Fragment of PSA variant product”—a sequence which is the same as part of but not all of the amino acid sequence of the PSA variant product,

“Fragments of PSA variant nucleic acid sequence” a continuous portion, preferably of about 20 nucleic acid sequences of the PSA variant nucleic acid sequence (see below), which sequence does not appear in the original PSA.

“Conservative substitution”—refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

“Chemically modified”—when referring to the product of the invention, means a product (protein) where at least one of its amino acid resides is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

“Biologically active”—refers to a PSA variant product which has the ability to serve as a marker of cancer, of predisposition to cancer, or of malignancy of a tumor.

“Immunologically active” defines the capability of a natural, recombinant or synthetic PSA variant product, or any fragment thereof, to induce a specific immune response in appropriate animals or cells and to bind with specific antibodies. Thus, for example, a biologically active fragment of PSA variant product denotes a fragment which retains some or all of the biological properties of the PSA variant product, e.g the ability to serve as a marker for prostate cancer; an immunologically active fragment is a fragment which can bind specific anti-PSA variant product antibodies or “distinguishing antibodies” (see below) which can elicit an immune response which will generate such antibodies or cause proliferation of PSA variant product-specific immune cells. The fragment will also be denoted hereinafter as “distinguishing amino acid sequence”.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from MacVector (™), operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence).

“Having at least X% identity”—with respect to two amino acid or nucleic acid sequence sequences, refers to the percentage of residues that are identical in the two sequences when the sequences are optimally aligned. Thus, 90% amino acid sequence identity means that 90% of the amino acids in two or more optimally aligned polypeptide sequences are identical.

“Isolated nucleic acid molecule having an PSA variant nucleic acid sequence”—is a nucleic acid molecule that includes the coding PSA variant nucleic acid sequences. Said isolated nucleic acid molecule may include the PSA variant nucleic acid sequence as an independent insert; may include the PSA variant nucleic acid sequence fused to an additional coding sequences, encoding together a fusion protein in which the PSA variant coding sequence is the dominant coding sequence (for example, the additional coding sequence may code for a signal peptide); the PSA variant nucleic acid sequence may be in combination with non-coding sequences, e.g., introns or control elements, such as promoter and terminator elements or 5′ and/or 3′ untranslated regions, effective for expression of the coding sequence in a suitable host; or may be a vector in which the PSA variant protein coding sequence is a heterologous.

“Expression vector”—refers to vectors that have the ability to incorporate and express heterologous DNA fragments in a foreign cell. Many prokaryotic and eukatyotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid Sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Antibody”—refers to IgG, IgM, IgD, IgA, and IgG antibody. The definition includes polyclonal antibodies or monoclonal antibodies. This term refers to whole antibodies or fragments of the antibodies comprising the antigen-binding domain of the anti-PSA variant product antibodies, e.g. antibodies without the Fc portion, single chain antibodies, fragments consisting of essentially only the variable, antigen-binding domain of the antibody, etc.

“Distinguishing antibody”—an antibody capable of binding only to the novel PSA variant product of the invention while not binding to the original PSA product, i.e. an antibody recognizing an additional amino acid sequence which appears only in the variant product of the invention and not in the original PSA sequence. This term may also refer at times to antibodies which bind a sequence present in the original PSA and not present in the PSA variant product.

“Distinguishing amino acid sequence”—an amino acid sequence of at least two amino acids which are present only in the PSA variant of the invention and not in the original PSA of which are used to prepare the above distinguishing antibodies.

“Activator”—as used herein, refers to a molecule which mimics the effect of the natural PSA variant product or at times even increases or prolongs the duration of the biological activity of said product, as compared to that induced by the natural product. The mechanism may be by binding to the PSA variant receptor, by prolonging the lifetime of the PSA variant, by increasing the activity of the PSA variant on its target, by increasing the affinity of PSA variant to its receptor, etc. Activators may be polypeptides, nucleic acids, carbohydrates, lipids, or derivatives thereof, or any other molecules which can bind to and activate the PSA variant product.

“Deactivator”—refers to a molecule which modulates the activity of the PSA variant product in an opposite manner to that of the activator, by decreasing or shortening the duration of the biological activity of the PSA variant product. This may be done by blocking the binding of the PSA variant to its receptor, competitive or non competitive inhibitor, by causing rapid degradation of the PSA variant etc. Deactivators may be polypeptides, nucleic acids, carbohydrates, lipids, or derivatives thereof, or any other molecules which bind to and modulate the activity of said product.

“Treating a disease”—refers to administering a therapeutic substance effective to ameliorate symptoms associated with a disease, to lessen the severity or cure the disease, or to prevent the disease from occurring. In the context of the invention the disease is typically cancer and in particular prostate cancer, breast cancer, ovarian cancer, salivary gland cancer and lung cancer.

The term “Treating a disease” may also include, for example, use of a molecule, expressed in a specific disease or correlated to a disease, as an immunogen. Such use does not require prior knowledge of the molecule's functionality. There are several methodologies available, which make use of non-characterized antigens as stimulants of an immune response for the treatment of various diseases. For example, it is well known in the art that diverse therapeutic vaccines, including cancer vaccines, can be used through a variety of available methods to stimulate an immune response to a particular disease-related antigen (Slovin, S. F., Hematol. Oncol. Clin. North Am., 15: 477-496, 2001).

“Detection”—refers to a method of detection of a disease, such as prostate cancer, breast cancer, ovarian cancer, salivary gland cancer and lung cancer.

May be detection of an active disease or detection of a predisposition to a disease. By another alternative the detection may be capable of distinguishing between benign and malignant conditions. This term may also be used in connection with a method for evaluating the aggressiveness of a malignant state in order to correctly predict the prognosis of the patient, and in that case the detection may be used to assess the stage of the tumor.

“Probe”—the PSA variant nucleic acid sequence, or a sequence (including fragments) complementary therewith, when used to detect presence of other similar sequences in a sample. The detection is carried out by identification of hybridization complexes between the probe and the assayed sequence. The probe may be attached to a solid support or to a detectable label. The probe may be a fragment of any one of the SEQ ID NO: 1 to SEQ ID NO: 6 (including a fragment of the non-coding region) which is of sufficient length to hybridize to the PSA variants at a level significantly different from the binding to the original PSA sequence. The probes may also be used to detect the polymorphisms described in the nucleic acid for the purpose of determining predisposition to cancer, especially prostate cancer in healthy individuals, and for detecting loss of heterozigosity in prostate tissues as part of a malignant transformation. The probes may be used in any method of performing this assay, including primer-specific PCR, allele-specific oligonucleotide assay, restriction fragment length differences, and mini-sequencing.

“Targeting”—directing a compound or drug to a desired cell population. Targeting is carried out by conjugating to the compound or drug an agent capable of binding specifically to the desired cell population, while not binding to non-desired cell populations. A specific example is targeting cytotoxic drugs directed only to tumor cells, more specifically directed to prostate tumor cells, for example, by conjugating the drug to an antibody of the invention.

“Original PSA sequence”—the known sequence of PSA as appears in GenDank HSPSAR locus and Ace # X05332, and is depicted in SEQ ID NO: 17 and SEQ ID NO: 18, as well as to the known KLK-2 sequence as appears in GenBank KLK-2 (NM_{—}005551) and is depicted in SEQ ID NO: 19 and SEQ ID NO: 20.

### SUMMARY OF THE INVENTION

The present invention provides by its first aspect, a novel isolated nucleic acid molecule comprising or consisting of the coding sequence of any one of SEQ ID NO: 1 to SEQ ID NO: 6, fragments of said coding sequence having at least 20 nucleic acids, or a molecule comprising a sequence having at 90% identity to any one of SEQ ID NO: 1 to SEQ ID NO: 6. Preferably, the fragments should be such that they comprise sequences present in the PSA variants of the invention and not a sequence present in the original PSA (the term “original PSA” also includes the KLK-2 sequence).

These sequences are novel splice variants which results from alternative splicing of the original PSA sequence (this term according to the glossary refers also to the KLK-2sequence).

The present invention further provides a protein or polypeptide comprising or consisting of an amino acid sequence encoded by any of the above nucleic acid sequences, termed herein “PSA variant product”, for example, an amino acid sequence having the sequence as depicted in any one of SEQ ID NO: 7 to SEQ ID NO: 12 fragments of the above amino acid sequence having a length of at least 10 amino acids, in particular fragments comprising sequences which do not appear in the original PSA sequence, as well as homologues of the amino acid sequences SEQ ID NO: 7 to SEQ ID NO: 12 in which one or more of the amino acid residues has been substituted (by conservative or non-conservative substitution) added, deleted, or chemically modified.

The novel PSA variant products of the invention may have the same physiological activity as the original PSA peptide (this term refers also to the KLK-2 product) from which they are varied (although perhaps at a different level); may have an opposite physiological activity from the activity featured by the original peptide from which they are varied; may have a completely different, unrelated activity to the activity of the original from which they are varied; or alternatively may have no activity at all and this may lead to various diseases or pathological conditions.

The novel variants of the invention whether being nucleic acid or amino acid sequences may serve for detection purposes, i.e. their presence or level may be indicative of prostate cancer, predisposition to prostate cancer, malignancy of the cancer, stage of the cancer, or may be indicative to normal condition. Alternatively the ratio between the level of each variant and the level original PSA sequence from which it has been varied; the ratio of each variant to the or other variants; the total amount (sum) of two or more variants either by itself or compared to other variants; or the sum of two or more variants, may be indicative of cancer or predisposition to cancer in general, and prostate cancer or predisposition to prostate cancer in particular, as well as indicative of the malignancy of the cancer, its stage of development or of normal condition. The variants may be detected in blood or serum or in the prostate gland, the ovary, breast, lung or salivary glands, which may share gene properties with the prostate gland. The variant products may be soluble or membrane bound.

For example, for detection purposes, it is possible to establish differential expression of the various variants in various tissues. A certain variant may be expressed mainly in one tissue, while the original PSA sequence may be expressed mainly in another tissue such as the prostate. Understanding of the distribution of the variants in various tissues may be helpful in basic research, for understanding the physiological function of the genes as well as may help in targeting pharmaceuticals or developing pharmaceuticals.

The study of the variants may also be helpful to distinguish various stages in the life cycles of the same type of cells which may also be helpful for development of pharmaceuticals for various pathological conditions in which cell cycles is un-normal, notably cancer. For example, various stages in the development of prostate cancer may be characterized by expression, or change in level of individual PSA variants of the invention.

Thus the detection may by determination of the presence or the level of expression of the variant within a specific cell population, comprising said presence or level between various cell types in a tissue, between different tissues and between individuals.

The present invention further provides nucleic acid molecule comprising or consisting of a sequence which encodes the above amino acid sequences, (including the Fragments and analogs of the amino acid sequences). Due to the degenerative nature of the genetic code, a plurality of alternative nucleic acid sequences, beyond those of SEQ ID NO: 1 to SEQ ID NO: 6, can code for the amino acid sequences of the invention. Those alternative nucleic acid sequences which code for the amino acid sequences codes by any one of the sequence SEQ ID NO: 1 to SEQ ID NO: 6 are also an aspect of the of the present invention.

The present invention further provides expression vectors and cloning vectors comprising any of the above nucleic acid sequences, as well as host cells transfected by said vectors.

The present invention still further provides pharmaceutical compositions comprising, as an active ingredient, said nucleic acid molecules, said expression vectors, or said protein or polypeptide.

These pharmaceutical compositions are suitable for the treatment of diseases and pathological conditions, which can be ameliorated or cured by raising the level of the PSA variant product, for example for the treatment of prostate cancer, or for inhibiting the transformation from prostate hyperplasia to malignancy. By another aspect, the present invention provides a nucleic acid molecule comprising or consisting of a non-coding sequence which is complementary to that of any one of SEQ ID NO: 1 to SEQ ID NO: 6, or complementary to a sequence having at least 90% identity to said sequence or a fragment of said two sequences. Ihe complementary sequence may be a DNA sequence which hybridizes with any one of the SEQ of ID NO: 1 to SEQ ID NO: 6 or hybridizes to a portion of that sequence having a length sufficient to inhibit the transcription of the complementary sequence. The complementary sequence may be a DNA sequence which can be transcribed into an mRNA being an antisense to the mRNA transcribed from SEQ ID NO: 1 to SEQ ID NO: 6 or into an mRNA which is an antisense to a fragment of the mRNA transcribed from SEQ ID NO.: 1 to SEQ ID NO: 6 which has a length sufficient to hybridize with the mRNA transcribed from SEQ ID NO:. 1 to SEQ ID NO: 6, so as to inhibit its translation. The complementary sequence may also be the mRNA or the fragment of the mRNA itself.

The nucleic acids of the invention may be used for therapeutic or diagnostic applications for example for detection of the expression of PSA variant in various tissues which may be indicative to the presence of prostate cancer, indicative of pre-disposition to prostate cancer, as well as indicative of the malignancy and hence the prognosis of the prostate cancer. The variants of the invention may also be indicative of other types of cancer from glands binding physiological similarity to the prostate gland such as ovary, breast, lung and salivary gland.

The present invention also provides expression vectors comprising any one of the above defined complementary nucleic acid sequences and host cells transfected with said nucleic acid sequences or vectors, being complementary to those specified in the first aspect of the invention.

The invention also provides anti-PSA variant product antibodies, namely antibodies directed against the PSA variant product which specifically bind to said PSA variant product. Said antibodies are useful both for diagnostic and therapeutic purposes. For example said antibody may be used to detect the presence of prostate specific antigen-variant product in various tissues which may be indicative of the presence of prostate cancer of a predisposition for having prostate cancer, or of the malignancy of prostate cancer.

The present invention further concerns distinguishing antibodies which can bind only to a sequence present in the variants of the invention which is not present (as a continuous sequence) in the original PSA sequence. The present invention further concerns amino acid sequences for producing said distinguishing antibodies termed “distinguishing amino acid sequences” which are sequences present in the novel PSA variant and not present (as a continuous sequence) in the original PSA An example of such a sequence is the sequence of positions 33-51 in SEQ ID NO: 7 being:

Cys-Gln-Ala-Glu-Leu-Ser-Pro-Pro-Thr-Gln-His-Pro-Ser-Pro-Asp-Arg-Glu-Leu

The present invention also provides pharmaceutical compositions comprising, as an active ingredient, the nucleic acid molecules which comprise or consist of said complementary sequences, or of a vector comprising said complementary sequences. Alternatively, the pharmaceutical composition can comprise, as an active ingredient, said anti-PSA variant product antibodies, or said distinguishing antibodies.

The pharmaceutical compositions comprising said anti-PSA variant product antibodies, said distinguishing antibodies or the nucleic acid molecule comprising said complementary sequence, are suitable for the treatment of diseases and pathological conditions where a therapeutically beneficial effect may be achieved by neutralizing at least one of the PSA variants or decreasing the amount of the PSA variant product or blocking its binding to the receptor, for example, by the neutralizing effect of the antibodies, or by the effect of the antisense mRNA in decreasing expression level of the PSA variant product. An example of such a disease is prostate cancer. Furthermore, where the PSA variant is membrane bound, the anti-PSA variant antibodies may be used to target cytotoxic or cytostatic compounds to the tumor cells, in particular to prostate tumor cells. Since PSA variants may be produced specifically by prostate tumor cells, (and not normal prostate cells) and since this protein may be membrane associated, conjugates of anti-PSA variant antibodies and a drug can be targeted only to tumor cells and not harm healthy cells.

According to the third aspect of the invention the present invention provides methods for detecting the level of the transcript (mRNA) of said PSA variant product in a body fluid sample, or in a specific tissue sample, for example by use of probes comprising or consisting of said sequences (which may be a coding or uncoding sequence), as well as methods for detecting levels of expression of said product in tissue, e.g. by the use of antibodies capable of specifically reacting with the above amino acid sequences.

The method, according to this latter aspect, for detection of a nucleic acid sequence which encodes the PSA variant product in a biological sample, comprises the steps of:

(a) providing a probe comprising at least one of the nucleic acid sequence defined above;

(b) contacting the biological sample with said probe under conditions allowing hybridization of nucleic acid sequences thereby enabling formation of hybridization complexes;

(c) detecting hybridization complexes, wherein the presence of the complex indicates the presence of nucleic acid sequence encoding the PSA variant product in the biological sample.

The method as described above is qualitative, i.e. indicates whether the transcript is present in or absent from the sample. The method can also be quantitative, by determining the level of hybridization complexes and then calibrating said levels to determining levels of transcripts of the desired PSA variant in the sample.

Both qualitative and quantitative determination methods can be used for diagnostic, prognostic and therapy planning purposes.

By a preferred embodiment the probe is part of a nucleic acid chip used for detection purposes, i.e. the probe is a part of an array of probes each present in a known location on a solid support.

As indicated above the method may be utilized for detecting the presence of prostate cancer, detecting predisposition to prostate cancer, or evaluating the malignancy of prostate cancer, or assessing the development stage of the cancer.

The nucleic acid sequence used in the above method may be a DNA sequence, an RNA sequence, etc; it may be a coding or a sequence, or a non-coding sequence, or a sequence complementary thereto (for respective detection of RNA transcripts or coding-DNA sequences). By quantization of the level of hybridization complexes and calibrating the quantified results it is possible also to detect the level of the transcript in the sample.

The probes of the invention may be used to detect polymorphisms (in a specific individual or while screening a population) specifically for pre-disposition to cancer (especially prostate cancer) and loss-of-heterozigosity may be important for monitoring the development of the disease. Detection of disease predisposition or loss of heterozigosity in prostate tissue may be performed on either the coding or non-coding DNA sequence. One example of such a test is the determination of the exact sequence before position 5620 in GenBank HUMPSANTIG/1257 of SEQ ID 1 5620 (which is non coding and which contains an additional inserted A as compared to the native PSA), or testing a possible A to G substitution in position 5573 of HUMPSANTIG/1210 of SEQ ID 1. Both these sites may be indicative of cancer risk and useful in prognosis.

Methods for detecting mutations in the region coding for the PSA variant product are also provided, which may be methods carried-out in a binary fashion, namely merely detecting whether there is any mismatches between the normal PSA variant nucleic acid sequence and the one present in the sample, or carried-out by specifically detecting the nature and location of the mutation.

The present invention also concerns a method for detecting PSA variant product in a biological sample, comprising the steps of:

(a) contacting with said biological sample the antibody of the invention, thereby forming an antibody-antigen complex; and

(b) detecting said antibody-antigen complex

wherein the presence of said antibody-antigen complex correlates with the presence of PSA variant product in said biological sample.

As indicated above, the method can be quantitized to determine the level or the amount of the PSA variant in the sample, alone or in comparison to the level of the original PSA amino acid sequence from which it was varied, and qualitative and quantitative results may be used for diagnostic, prognostic and therapy planning purposes.

By yet another aspect the invention also provides a method for identifying candidate compounds capable of binding to the PSA variant product and modulating its activity (being either activators or deactivators). The method includes:

(i) providing a protein or polypeptide comprising an amino acid sequence substantially as depicted in any one of SEQ ID NO: 7 to SEQ ID NO: 12, or a fragment of such a sequence;

(ii) contacting a candidate compound with said amino acid sequence;

(iii) measuring the physiological effect of said candidate compound on the activity of the amino acid sequences and selecting those compounds which show a significant effect on said physiological activity.

The activity of the amino acid which should be changed by the modulator (being either the activator or deactivator) may be for example the binding of the amino acid (PSA variant product) to its native, receptor. Any modulator which changes such an activity has an intersecting potential.

The present invention also concerns compounds identified by the above methods described above, which compound may either be an activator of the serotonin-receptor like product or a deactivator thereof.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows multiple alignment between the original PSA sequence (SEQ ID NO: 14) and polypetides encoded by the sequences of the invention (PSAL-O is SEQ ID NO: 1; PSAL-1 is SEQ ID NO: 2, PSAL-2 is SEQ ID NO: 3; PSAL-5 is SEQ ID NO: 4 and PSAL-6 is SEQ ID NO: 5);

FIG. 2 shows a multiple alignment between the 5 PSA splice variant products, encoded by SEQ ID NOs: 1-5, as described in FIG. 3, and the direct translation of the genomic PSA region, depicted here as HUMPSANTIG (SEQ ID NO: 13);

FIG. 3 shows the specific region (signal peptide) (SEQ ID NO: 15) of the original KLK-2 (accession number NM_{—}005551) that is common with the splice variant of KLK-2 (SEQ ID NO: 12);

FIG. 4 shows a schematic representation of the common locus of the KLK-2 of PSA genes;

FIG. 5 shows a Northern Blot analysis of RNA obtained from various tissues and tested with probes for PSA (FIG. 5A) and probes obtained from SEQ ID NO: 2, (termed PSALM in the Figure) (FIG. 5B);

FIG. 6 shows a Western Blot analysis of proteins obtained from prostate glands of several patients tested for PSA protein (left) and the PSA variant protein depicted in SEQ ID NO: 7 (right);

FIG. 7 shows immuno-histochemical labeling of human prostate gland with serum of rabbit immunized with PSAL variant peptide of the invention, corresponding to SEQ ID NO: 7, (right) or unimmunized rabbit;

FIG. 8 shows a cross-section of in-situ hybridization of sense and anti-sense probes of PSA (termed “PSALM”), derived from sequence depicted as SEQ ID NO: 2, to tissue obtained from a prostate cancer;

FIG. 9 shows a Western blot analysis of 2 different serum-samples tested for PSA protein PSA (termed “PSALM”), using anti-PSALM. The results indicate that the PSA, corresponding to SEQ ID NO: 7, is secreted to the serum. In the right lane there is a recombinant PSA;

FIG. 10 shows immuno-histochemical labeling of human prostate gland with serum of rabbit immunized with the peptide of the invention (derived from alternative splicing of the KLK-2 gene) (FIG. 10A) or immunized rabbit (FIG. 10B); and

FIG. 11 shows a Western blot analysis of proteins obtained from prostate glands of 2 different patients (prostate #1 and #2), and from other tissues tested for the protein of the invention derived from the KLK-2 gene, with anti KLM.

FIG. 12 shows the specific region (signal peptide) of the original PSA (SEQ ID NO: 17) that is common with all the splice variants of PSA (SEQ ID NOs: 1-5).

FIG. 13 shows Northen blot analysis demonstrating the hormonal regulation of PSA (SEQ ID NO: 17) and PSALM (SEQ ID NO: 1) mRNA expression.

FIG. 14A shows detection of Myc-tagged PSALM (SEQ ID NO: 7) protein, and FIG. 14B shows detection of Myc-tagged KLM (SEQ ID NO: 12) protein, demonstrated in transfected LNCaP cells. The recombinant proteins expression was demonstrated in cell lysates by immunodetection, and the secretion of the recombinant proteins was verified by immunoprecipitation of spent media followed by immunodetection, as described in Example VIIA.

FIG. 15 shows immunoblot analysis, demonstrating the secretion of KLM protein (SEQ ID NO: 12) to seminal fluid and its expression in Benign Prostatic Hyperplasia (BPH), as described in Example VIIB.

FIG. 16 shows a western blot analysis, demonstrating expression of endogenous PSA (SEQ ID NO: 18) (FIG. 16A), KLM (SEQ ID NO: 12) (FIG. 16B), and PSA-LM (SEQ ID NO: 7) (FIG. 16C) in various tissue samples and cell lines. The figure demonstrates the expression of the proteins in lung tissue, as described in Example VIII.

FIG. 17 shows immuno-histochemical labeling of lung sections using anti PSA-LM (SEQ ID NO: 7) monoclonal antibody. Cytoplasmic staining within the epithelial cells of the bronchus wall (A), the epithelial cells lining the submucosal glands (A and B), moderately differentiated adenocarcinoma (C) and squamous cell carcinoma (D) using anti PSA-LM monoclonal antibody demonstrates specific staining in normal epithelium (FIGS. A and B) and neoplastic epithelial cells (FIGS. C and D).

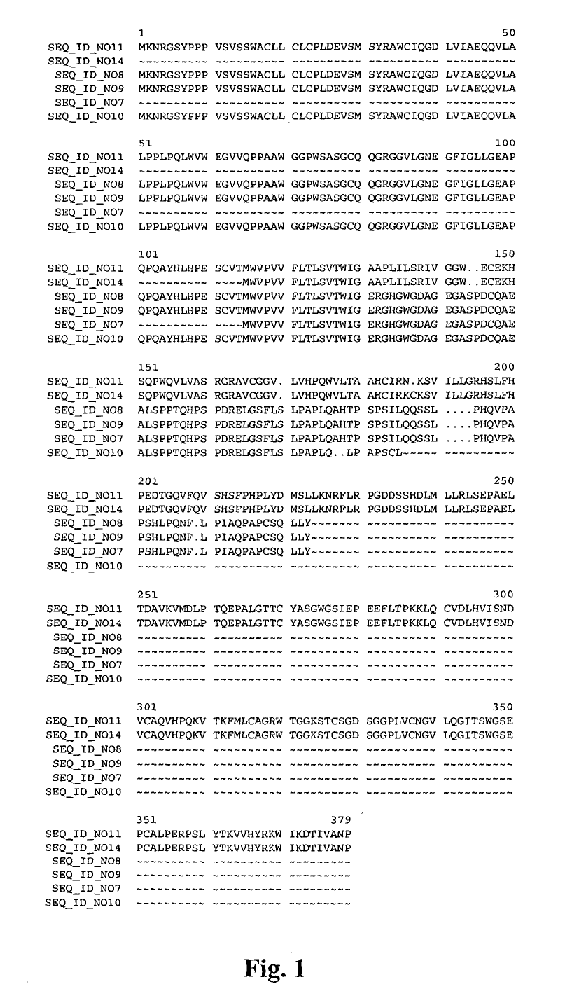

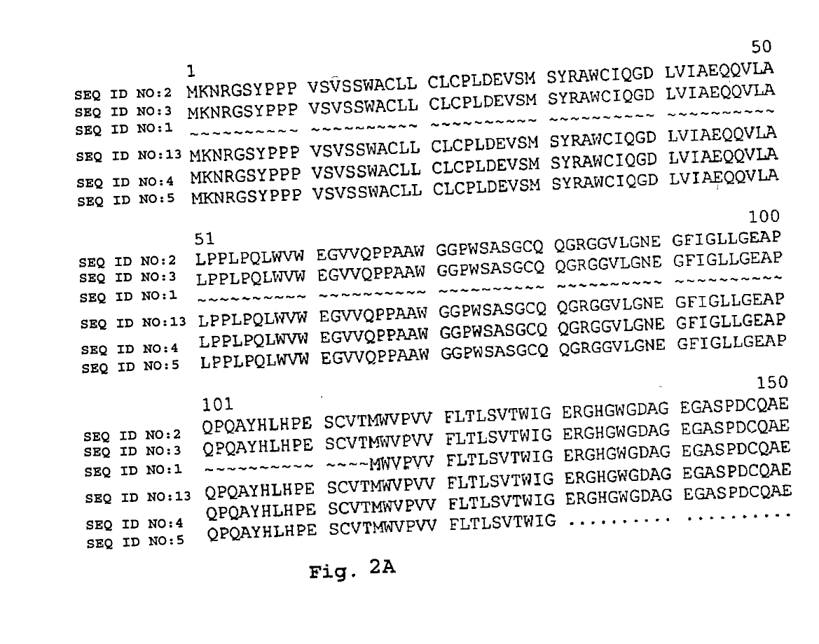

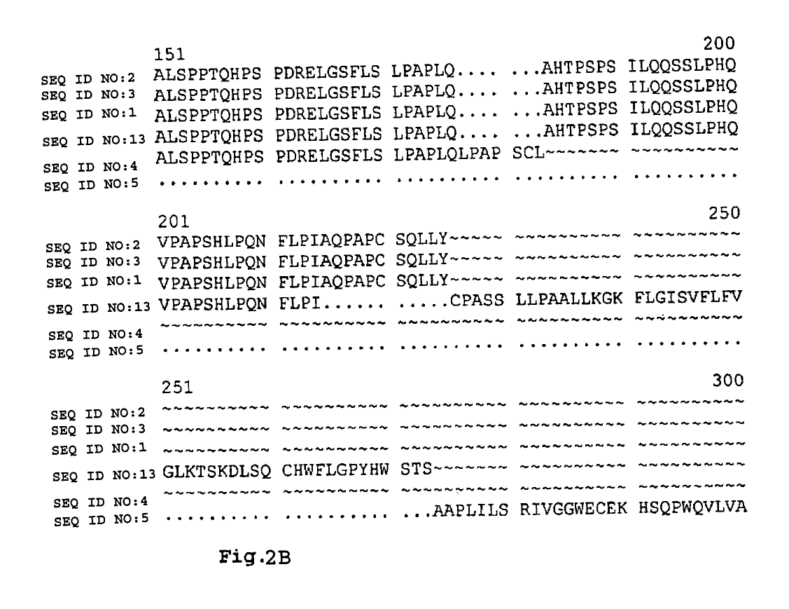

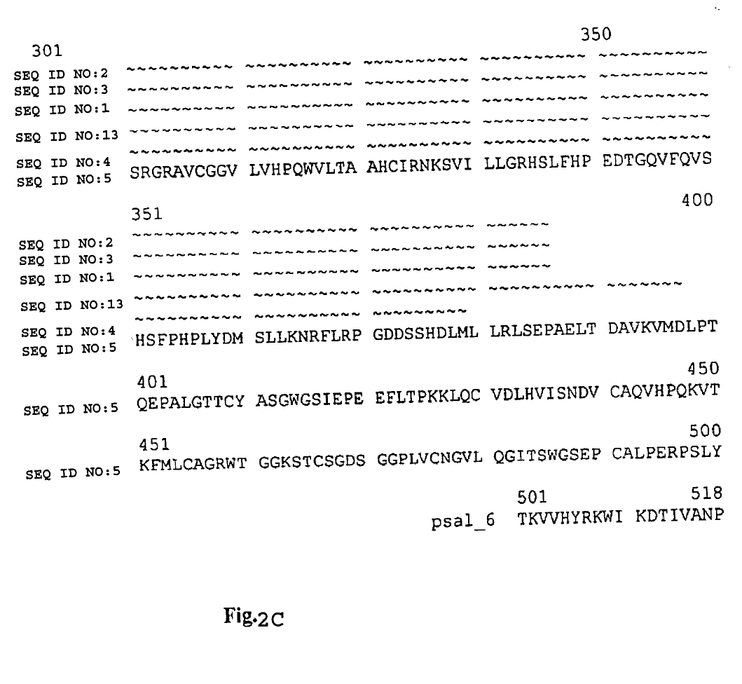

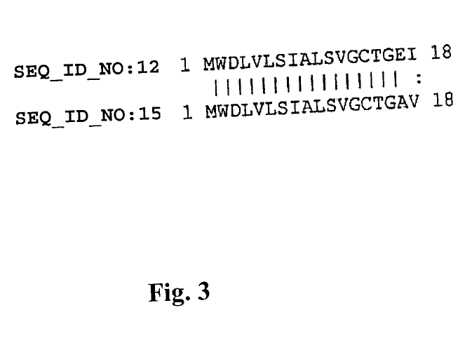

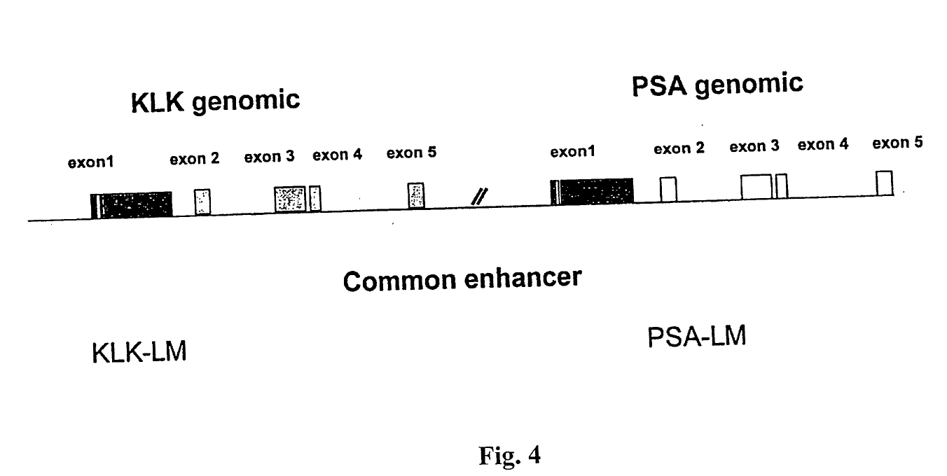

### DETAILED DESCRIPTION OF A PREFERRED EMBODIMENT

### EXAMPLE I

PSA Variant—Nucleic Acid Sequence

The nucleic acid sequences of the invention include nucleic acid sequences which encode PSA variant product and fragments and analogs thereof. The nucleic acid sequences may alternatively be sequences complementary to the above coding sequence, or to a region of said coding sequence. The length of the complementary sequence is sufficient to avoid the expression of the coding sequence. The nucleic acid sequences may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand. The nucleic acid sequences may also both include dNTPs, rNTPs as well as non naturally occurring sequences. The sequence may also be a part of a hybrid between an amino acid sequence and a nucleic acid sequence.

In a general embodiment, the nucleic acid sequence has at least 70%, preferably 80% or 90% sequence identity with the sequence identified as SEQ ID NO; 1 to SEQ ID NO: 6.

The nucleic acid sequences may include the coding sequence by itself. By another alternative the coding region may be in combination with additional coding sequences, such as those coding for fusion protein or signal peptides, in combination with non-coding sequences, such as introns and control elements, promoter and terminator elements or 5′ and/or 3′ untranslated regions, effective for expression of the coding sequence in a suitable host, and/or in a vector or host environment in which the PSA variant nucleic acid sequence is introduced as a heterologous sequence.

The nucleic acid sequences of the present invention may also have the product coding sequence fused in-frame to a marker sequence which allows for purification of the PSA variant product. The marker sequence may be, for example, a hexahistidine tag to provide for purification of the mature polypeptide fused to the marker in the case of a bacterial host, or, the marker sequence may be a hemagglutinin (HA) tag when a mammalian host, e.g. COS-7 cells, is used. The HA tag corresponds to an epitope derived from the influenza hemagglutinin protein (Wilson, I., et al. Cell 37:767 (1984)).

Also included in the scope of the invention are fragments also referred to herein as oligonucleotides, typically having at least 20 bases, preferably 20-30 bases corresponding to a region of the coding-sequence nucleic acid sequence. The fragments may be used as probes, primers, and when complementary also as antisense agents, and the like, according to known methods,

As indicated above, the nucleic acid sequence may be substantially a depicted in SEQ ID NO: 1 to SEQ ID NO: 6 or fragments thereof or sequences having at least 70%, preferably 70-80%, most preferably 90% identity to the above sequence. Alternatively, due to the degenerative nature of the genetic code, the sequence may be a sequence coding the amino acid sequence of SEQ ID NO: 6 to SEQ ID NO: 12, or fragments or analogs of said amino acid sequence.

A. Preparation of Nucleic Acid Sequences

The nucleic acid sequences may be obtained by screening cDNA libraries using oligonucleotide probes which can hybridize to or PCR-amplify nucleic acid sequences which encode the PSA variant products disclosed above. EDNA libraries prepared from a variety of tissues are commercially available and procedures for screening and isolating cDNA clones are well-known to those of skill in the art. Such techniques are described in, for example, Sambrook et al. (1989) Molecular Cloning: A Laboratory Manual (2nd Edition), Cold Spring Harbor Press, Plainview, N.Y. and Ausubel F M et al. (1989) Current Protocols in Molecular Biology, John Wiley & Sons, New York, N.Y.

The nucleic acid sequences may be extended to obtain upstream and downstream sequences such as promoters, regulatory elements, and 5′ and 3′ untranslated regions (UTRs). Extension of the available transcript sequence may be performed by numerous methods known to those of skill in the art, such as PCR or primer extension (Sambrook et al., supra), or by the RACE method using, for example, the MARATHON RACE kit (Clontech, Cat. # K1802-1).

Alternatively, the technique of “restriction-site” PCR (Gobinda et al. PCR Methods Applic. 2:318-22, (1993)), which uses universal primers to retrieve flanking sequence adjacent a known locus, may be employed. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia, T. et al., Nucleic Acids Res. 16:8186, (1988)). The primers may be designed using OLIGO(R) 4.06 Primer Analysis Software (1992; National Biosciences Inc, Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Capture PCR (Lagerstrom, M. et al., PCR Methods Applic. 1:111-19, (1991)) is a method for PCR amplification of DNA fragments adjacent to a known sequence in human and yeast artificial chromosome DNA. Capture PCR also requires multiple restriction enzyme digestions and ligations to place an engineered double-stranded sequence into a flanking part of the DNA molecule before PCR.

Another method which may be used to retrieve flanking sequences is that of Parker, J. D., et al., Nucleic Acids Res., 19:3055-60, (1991)). Additionally, one can use PCR, nested primers and PromoterFinder™ libraries to “walk in” genomic DNA (PromoterFinder™; Clontech, Palo Alto, Calif.). This process avoids the need to screen libraries and is useful in finding intron/exon junctions. Preferred libraries for screening for full length cDNAs are ones that have been size-selected to include larger cDNAs. Also, random primed libraries are preferred in that they will contain more sequences which contain the 5′ and upstream regions of genes.

A randomly primed library may be particularly useful if an oligo d(T) library does not yield a full-length cDNA. Genomic libraries are useful for extension into the 5′ nontranslated regulatory region.

The nucleic acid sequences and oligonucleotides of the invention can also be prepared by solid-phase methods, according to known synthetic methods. Typically, fragments of up to about 100 bases are individually synthesized, then joined to form continuous sequences up to several hundred bases.

B. Use of PSA Variant Nucleic Acid Sequence for the Production of PSA Variant Products

In accordance with the present invention, nucleic acid sequences specified above may be used as recombinant DNA molecules that direct the expression of PSA variant products.

As will be understood by those of skill in the art, it may be advantageous to produce PSA variant product-encoding nucleotide sequences possessing codons other than those which appear in any one of SEQ ID NO: 1 to SEQ ID NO: 6 which are those which naturally occur in the human genome. Codons preferred by a particular prokaryotic or eukaryotic host (Murray, E. et al. Nuc Acids Res., 17:477-508, (1989)) can be selected, for example, to increase the rate of PSA variant product expression or to produce recombinant RNA transcripts having desirable properties, such as a longer half-life, than transcripts produced from naturally occurring sequence.

The nucleic acid sequences of the present invention can be engineered in order to alter a PSA variant product coding sequence for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, to produce splice variant, etc.

The present invention also includes recombinant constructs comprising one or more of the sequences as broadly described above. The constructs comprise a vector, such as a plasmid or viral vector, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al., (supra).

The present invention also relates to host cells which are genetically engineered with vectors of the invention, and the production of the product of the invention by recombinant techniques. Host cells are genetically engineered (i.e., transduced, transformed or transfected) with the vectors of this invention which may be, for example, cloning vectors or expression vectors. The vector may be, for example, in the form of a plasmid, a viral particle, a phage, etc. The engineered host cells can be cultured in conventional nutrient media modified as appropriate for activating promoters, selecting transformants or amplifying the expression of the PSA variant nucleic acid sequence. The culture conditions, such as temperature, pH and the like, are those previously used with the host cell selected for expression, and will be apparent to those skilled in the art.

The nucleic acid sequences of the present invention may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence promoter) to direct mRNA synthesis. Examples of such promoters include: LR or SV40 promoter, the E.coli lac or trp promoter, the phage lambda PL promoter, and other promoters known to control expression of genes in prokaryotic or eukaryotic cells or their viruses. The expression vector also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited by the host cells employed.

In bacterial systems, a number of expression vectors may be selected depending upon the use intended for the PSA variant product. For example, when large quantities of PSA variant product are needed for the induction of antibodies, vectors which direct high level expression of fusion proteins that are readily purified may be desirable. Such vectors include, but are not limited to, multifunctional E. coli cloning and expression vectors such as BLUESCRIPT(®) (Stratagene), in which the PSA variant polypeptide coding sequence may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); and the like

In the yeast Saccharomyces cerevisiae a number of vectors containing constitutive or inducible promoters such as alpha factor, alcohol oxidase and PGH may be used. For reviews, see Ausubel et al (supra) and Grant et al., (Methods in Enzymology 153:516-544, (1987)).

In cases where plant expression vectors are used, the expression of a sequence encoding PSA variant product may be driven by any of a number of promoters. For example, viral promoters such as the 35S and 19S promoters of CaMV (Brisson et al, Nature 310:511-514. (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or beat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

PSA variant product may also be expressed in an insect system. In one such system, Autographa californica nuclear polyhedrosis virus (AcNPV) is used as a vector to express foreign genes in Spodoptera frugiperda cells or in Trichoplusia larvae. The PSA variant product coding sequence may be cloned into a nonessential region of the virus, such as the polyhedrin gene, and placed under control of the polyhedrin promoter. Successful insertion of PSA variant coding sequence will render the polyhedrin gene inactive and produce recombinant virus lacking coat protein coat. The recombinant viruses are then used to infect S. frugiperda cells or Trichoplusia larvae in which PSA variant protein is expressed (Smith et al., J. Virol. 46:584, (1983); Engelhard, E. K. et al., Proc. Nat. Acad. Sci. 91:3224-7, (1994)).

In mammalian host cells, a number of viral-based expression systems may be utilized. In cases where an adenovirus is used as an expression vector, a PSA variant product coding sequence may be ligated into an adenovirus transcription/translation complex consisting of the late promoter and tripartite leader sequence. Insertion in a nonessential E1 or E3 region of the viral genome will result in a viable virus capable of expressing PSA variant protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells.

Specific initiation signals may also be required for efficient translation of a PSA variant protein coding sequence. These signals include the ATG initiation codon and adjacent sequences. In cases where PSA variant product coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Differ., 20:125-62, (1994); Bittner et al., Methods in Enzymol 153:516-544, (1987)).

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express PSA variant product may be transformed using expression vectors which contain viral origins of replication or endogenous expression elements and a selectable marker gene. Following the introduction of the vector, cells may be allowed to grow for 1-2 days in an enriched media before they are switched to selective media, The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt- cells, respectively. Also, antimetabolite, antibiotic or herbicide resistance can be used as the basis for selection; for example, dhfr which confers; resistance to methotrexate (Wigler M,, et al., Proc. Natl. Acad. Sci. 77:3567-70, (1980)); npt, which confers resistance to the aminoglycosides neomycin and G-418 (Colbere-Garapin, F. et al., J. Mol. Biol., 150:1-14, (1981)) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci. 85:8047-51, (1988)). The use of visible markers has gained popularity with such markers as anthocyanins, beta-glucuronidase and its substrate, GUS, and luciferase and its substrates, luciferin and ATP, being widely used not only to identify transfomants, but also to quantify the amount of transient or stable protein expression attributable to a specific vector system (Rhodes, C. A. et. al., Methods Mol. Biol., 55:121-131, (1995)).

Host cells transformed with a nucleotide sequence encoding PSA variant product may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding PSA variant product can be designed with signal sequences which direct secretion of PSA variant product through a prokaryotic or eukaryotic cell membrane.

PSA variant product may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.). The inclusion of a protease-cleavable polypeptide linker sequence between the purification domain and PSA variant protein is useful to facilitate purification.

One such expression vector provides for expression of a fusion protein compromising a PSA variant polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating PSA variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

The PSA variant products can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

C. Diagnostic Applications Utilizing Nucleic Acid Sequences

The nucleic acid sequences of the present invention may be used for a variety of diagnostic purposes. The nucleic acid sequences may be used to detect and quantitate expression of PSA variant in patient's cells, e.g. biopsied tissues, by detecting the presence of mRNA coding for PSA variant product. Alternatively, the assay may be used to detect free PSA variant in the serum or blood. This assay typically involves obtaining total mRNA from the tissue or serum and contacting the mRNA with a nucleic acid probe. The probe is a nucleic acid molecule of at least 20 nucleotides, preferably 20-30 nucleotides, capable of specifically hybridizing with a sequence included within the sequence of a nucleic acid molecule encoding PSA variant under hybridizing conditions, detecting the presence of mRNA hybridized to the probe, and thereby detecting the expression of PSA variant. This assay can be used to distinguish between absence, presence, and excess expression of PSA variant product and to monitor levels of PSA variant expression during therapeutic intervention.

The invention also contemplates the use of the nucleic acid sequences as a diagnostic for diseases resulting from inherited defective PSA variant sequences. These sequences can be detected by comparing the sequences of the defective (i.e., mutant) PSA variant coding region with that of a normal coding region. Association of the sequence coding for mutant PSA variant product with abnormal PSA variant product activity may be verified. In addition, sequences encoding mutant PSA variant products can be inserted into a suitable vector for expression in a functional assay system (e.g., colorimetric assay, complementation experiments in a PSA variant protein deficient strain of HEK293 cells) as yet another means to verify or identify mutations. Once mutant genes have been identified, one can then screen populations of interest for carriers of the mutant gene.

Individuals carrying mutations in the nucleic acid sequence of the present invention may be detected at the DNA level by a variety of techniques. Nucleic acids used for diagnosis may be obtained from a patient's cells, including but not limited to such as from blood, urine, saliva, placenta, tissue biopsy and autopsy material and in particular tissue obtained from the prostate gland. Genomic DNA may be used directly for detection or may be amplified enzymatically by using PCR (Saiki, et al., Nature 324:163-166, (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype.

Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and S1 protection or the chemical cleavage method (e.g. Cotton, et al Proc. Natl. Acad. Sci. USA, 85:4397-4401, (1985)), or by differences in melting temperatures. “Molecular beacons” (Kostrikis L. G. et al., Science 279:1228-1229, (1998)), hairpin-shaped, single-stranded synthetic oligo-nucleotides containing probe sequences which are complementary to the nucleic acid of the present invention, may also be used to detect point mutations or other sequence changes as well as monitor expression levels of PSA variant product.

Another method for detecting mutations uses two DNA probes which are designed to hybridize to adjacent regions of a target, with abutting bases, where the region of known or suspected mutation(s) is at or near the abutting bases. The two probes may be joined at the abutting bases, e.g., in the presence of a ligase enzyme, but only if both probes are correctly base paired in the region of probe junction. The presence or absence of mutations is then detectable by the presence or absence of ligated probe.

Also suitable for detecting mutations in the PSA variant product coding sequence are oligonucleotide array methods based on sequencing by hybridization (SBH), as described, for example, in U.S. Pat. No. 5,547,839. In a typical method, the DNA target analyte is hybridized with an array of oligonucleotides formed on a microchip. The sequence of the target can then be “read” from the pattern of target binding to the array.

D. In situ Hybridization Using Probes of PSA

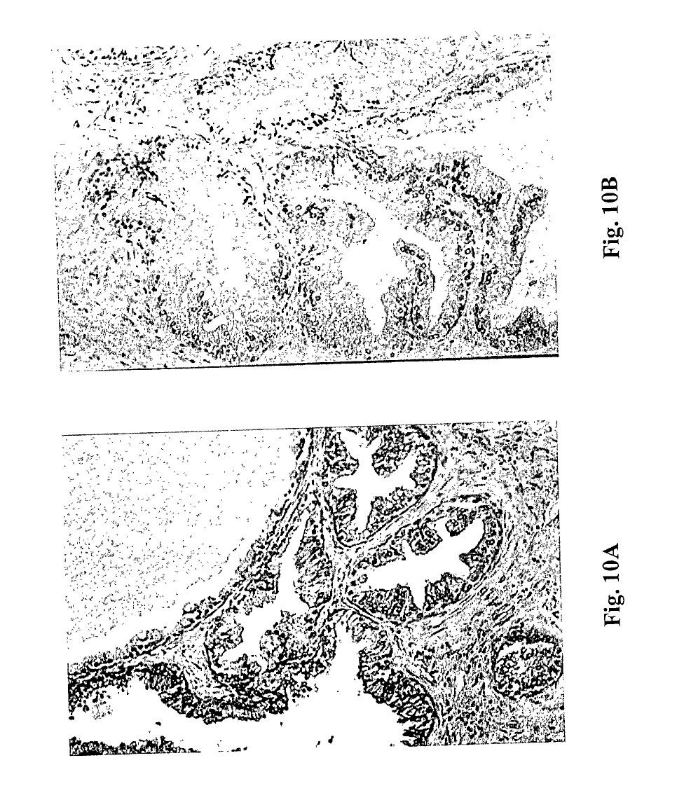

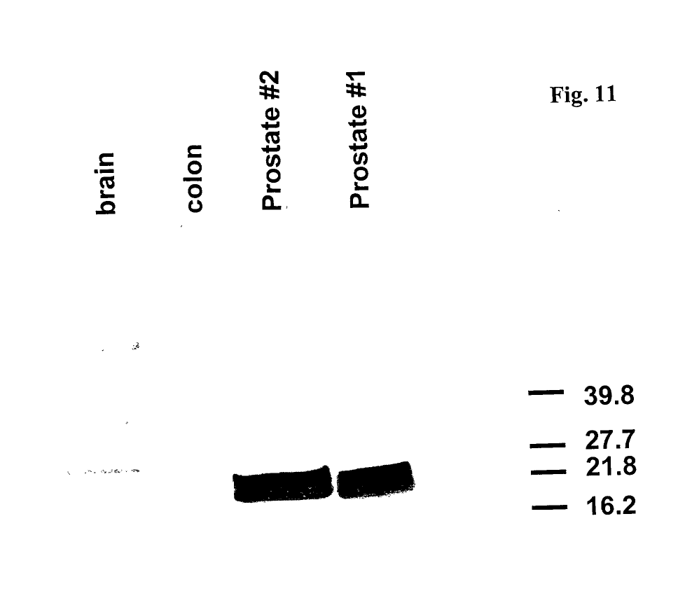

In-situ hybridisation was carried out according to the procedure described in the Boehringer-Mannheim's publication “Non-Radioative In-Situ Hybridization Application Manual”, 2^{nd} edition, 1996. Labelling was carried out according to Chapter 4, section V, and hybridization according to Chapter 5, section IV. Slides were prepared in paraffin and treated according to the procedures described in Chapter 2. The probe used was derived from the PSAL-1 sequence (SEQ ID NO: 2). The anti-sense probe was used to detect the presence of PSA variant mRNA, and the sense probe was used as control, Results in FIG. 10 indicates high-level expression of the PSA variant mRNA in prostate epithelial lumen cells.

E. Therapeutic Applications of Nucleic Acid Sequences

Nucleic acid sequences of the invention may also be used for therapeutic purposes. Turning first to the anti-PSA variant aspect, expression of PSA variant product may be modulated through antisense technology, which controls gene expression through hybridization of complementary nucleic acid sequences, i.e. antisense DNA or RNA, to the control, 5′ or regulatory regions of the gene encoding PSA variant product. For example, the 5′ coding portion of the nucleic acid sequence sequence which codes for the product of the present invention is used to design an antisense oligonucleotide of from about 10 to 40 base pairs in length. Oligonucleotides derived from the transcription start site, e.g. between positions −10 and +10 from the start site, are preferred. An antisense DNA oligonucleotide is designed to be complementary to a region of the nucleic acid sequence involved in transcription (Lee et al., Nucl. Acids, Res., 6:3073, (1979); Cooney et al., Science 241:456, (1988); and Dervan et al., Science 251:1360, (1991)), thereby preventing transcription and the production of the PSA variant products. An antisense RNA oligonucleotide hybridizes to the mRNA in vivo and blocks translation of the mRNA molecule into the PSA variant products (Okano J. Neurochem. 56:560, (1991)). The antisense constructs can be delivered to cells by procedures known in the art such that-the antisense RNA or DNA may be expressed in vivo. The antisense may be antisense mRNA or DNA sequence capable of coding such antisense mRNA. The antisense mRNA or the DNA coding thereof can be complementary to the full sequence of nucleic acid sequences coding to the PSA variant protein or to a fragment of such a sequence which is sufficient to inhibit production of a protein product.

Turning now to the PSA variant aspect, expression of PSA variant product may be increased by providing coding sequences for coding for said product under the control of suitable control elements ending its expression in the desired host.

The nucleic acid sequences of the invention may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The formulation should suit the mode of administration.

The polypeptides, and activator and deactivator compounds (see below), which are polypeptides, may also be employed in accordance with the present invention by expression of such polypeptides in vivo, which is often referred to as “gene therapy.” Cells from a patient may be engineered with a nucleic acid sequence (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by procedures known in the art. As known in the art, a producer cell for producing a retroviral particle containing RNA encoding the polypeptide of the present invention may be administered to a patient for engineering cells in vivo and expression of the polypeptide in Vivo. These and other methods for administering a product of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors mentioned above may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE50I, PA317, psi-2, psi-AM, PA12, T19-14X, VT-19-17-H2, psi-CRE, psi-CRIP, GP+E-86, GP+envAm12, and DAN cell lines as described in Miller (Human Gene Therpy, Vol. 1, pg. 5-14, (1990)). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

The genes introduced into cells may be placed under the control of inducible promoters, such as the radiation-inducible Egr-1 promoter, (Maceri, H. J., et al., Cancer Res., 56(19):4311 (1996)), to stimulate PSA variant production or antiserse inhibition in response to radiation, eg., radiation therapy for treating tumors.

F. Northern Blot Analysis

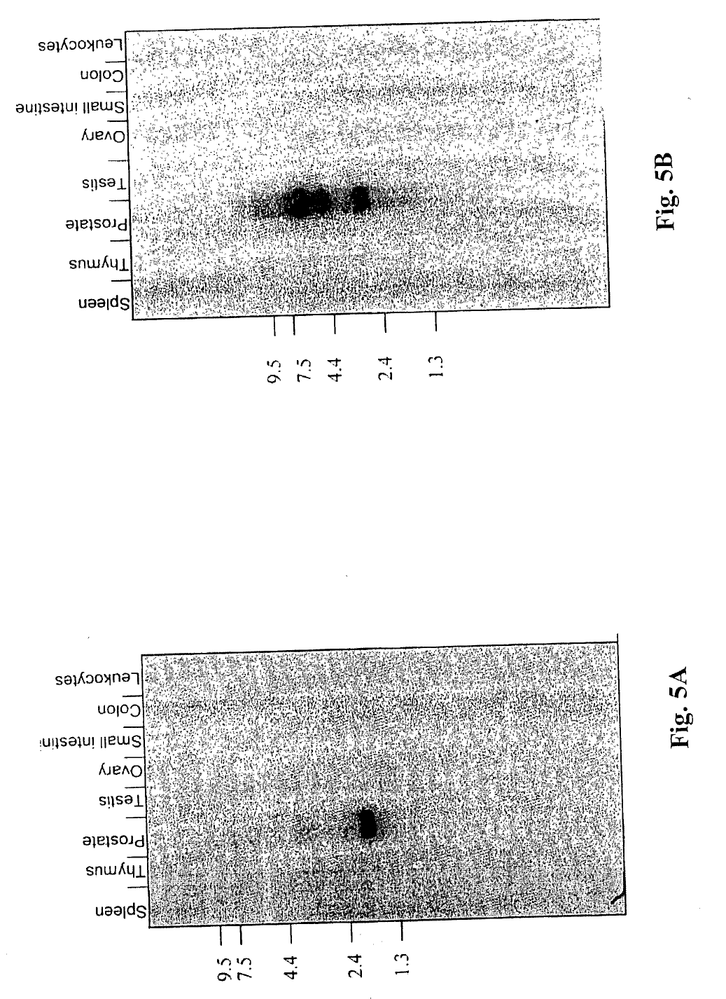

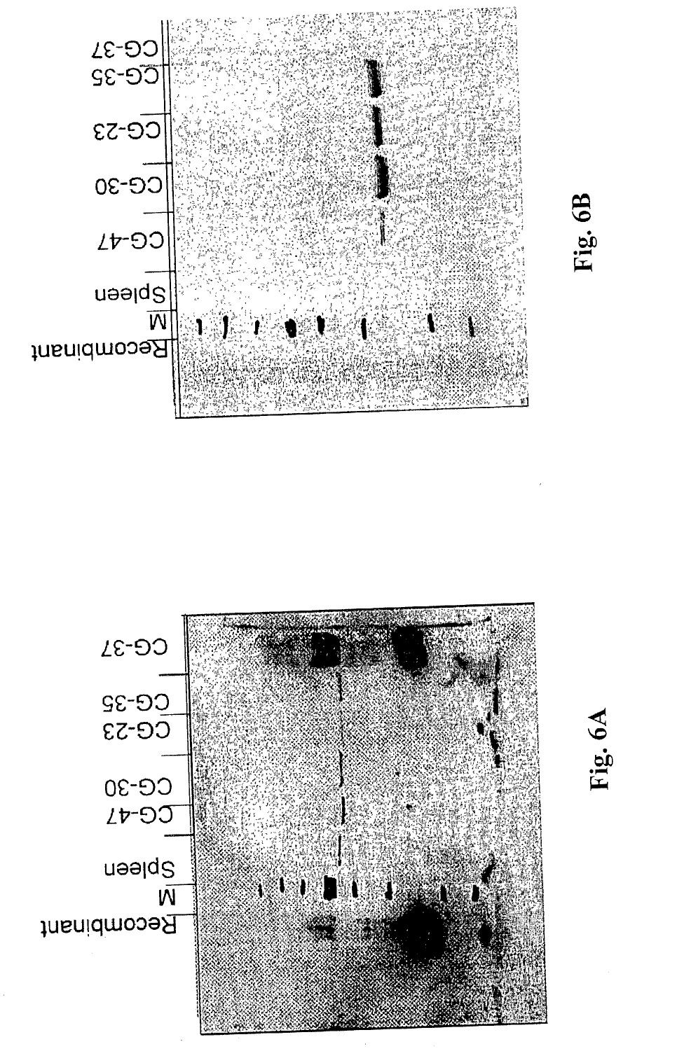

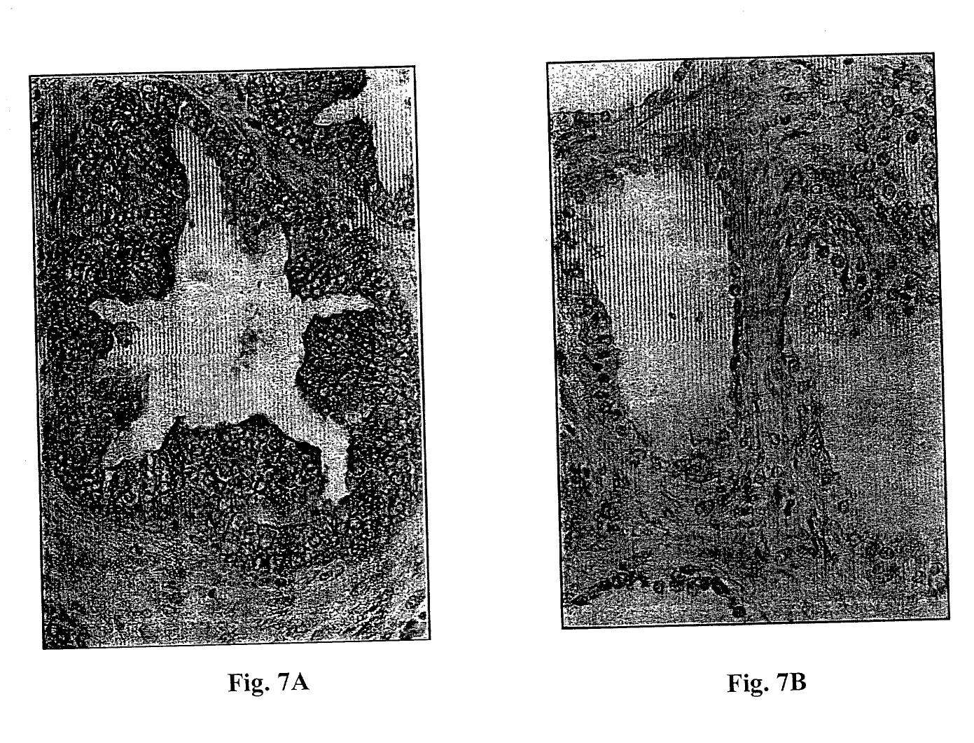

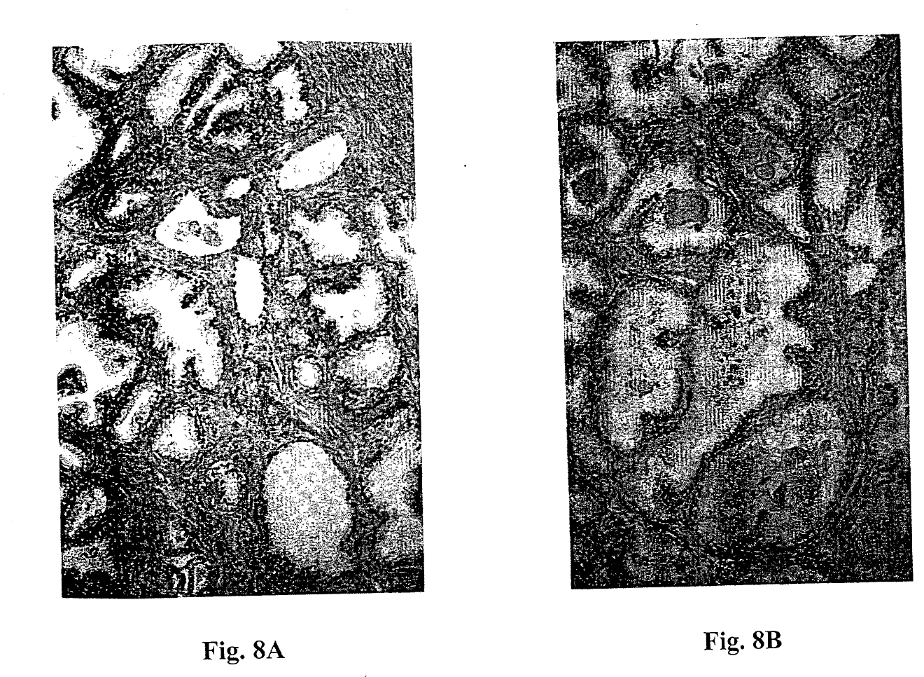

RNA samples were obtained from spleen, thymus, prostate, testis, ovary, small intestine, colon and leukocytes electrophoresed through a 1.5% agarose gel containing formaldehyde and transfered onto nylon (HYBOND-N, Amersham) paper (Thomas, 1980). Prehybridization was for 2 hours in a buffer containing 10% Dextrane Sulfate, 1M NaCl and 1% SDS, at 65° C. Hybridization was in the same buffer with 5×106 cpm of the appropriate probe at 65° C. for 18 hours. After one wash in 2×SSC, 0.1% SDS for 15 minutes at 65° C. and several washes in 0.2×SSC, 0.1% SDS at 65° C. the filter was exposed to an X-ray film. Phosphorimager analysis was performed as well. The results are shown in FIG. 5. As can be seen with the PSA probe (FIG. 5A), a single band was detected, while with the probe of the invention (termed “PSAL”) (derived from SEQ ID NO: 2, in the common region with SEQ IDs 3, 4, and 5) several bands were detected in prostate tissue, which indicates the presence of different PSA splice variants.

### EXAMPLE II

PSA Variant Product

The substantially purified PSA variant product of the invention has been defined above as the product coded from the nucleic acid sequence of the invention. Preferably the amino acid sequence is an amino acid sequence having at least 90% identity to the sequence identified as any one of SEQ ID NO: 7 to SEQ. ID NO. 12. The protein may be in mature and/or modified form, also as defined above, Also contemplated are protein fragments having at least 10 contiguous amino acid residues, preferably at least 10-20 residues, derived from the PSA variant protein.

The sequence variations are preferably those that are considered conserved substitutions, as defined above. Thus, for example, a protein with a sequence having at least 80% sequence identity with the protein identified as SEQ ID NO: 7 to SEQ ID NO: 12, preferably by utilizing conserved substitutions as defined above. In a more specific embodiment, the protein has or contains the sequence identified SEQ ID NO: 7 to SEQ ID NO: 12. The PSA variant product may be (i) one in which one or more of the amino acid residues in a sequence listed above are substituted with a conserved or non-conserved amino acid residue (preferably a conserved amino acid residue), or (ii) one in which one or more of the amino acid residues includes a substituent group, or (iii) one in which the PSA variant product is fused with another compound, such as a compound to increase the half-life of the protein (for example, polyethylene glycol (PEG)), or a moiety which serves as targeting means to direct the protein to its target tissue or target cell population (such as an antibody), or (iv) one in which additional amino acids are fused to the PSA variant product. Such fragments, variant and derivatives are deemed to be within the scope of those skilled in the art from the teachings herein.

A. Preparation of PSA Variant Product

Recombinant methods for producing and isolating the PSA variant product, and fragments of the protein are described above.

In addition to recombinant production, fragments and portions of PSA variant product may be produced by direct peptide synthesis using solid-phase techniques (cf. Stewart et al., (1969) Solid-Phase Peptide Synthesis, W H Freeman Co, San Francisco; Merrifield J., J. Am. Chem. Soc., 85:2149-2154, (1963)). In vitro peptide synthesis may be preformed using manual techniques or by automation. Automated synthesis may be achieved, for example, using Applied Biosystems 431A Peptide Synthesizer (Perkin Elmer, Foster City, Calif.) in accordance with the instructions provided by the manufacturer. Fragments of PSA variant product may be chemically synthesized separately and combined using chemical methods to produce the full length molecule.

B. Western Blot Analysis

Western Blot analysis was performed according to procedures well known in the art, that are described in the Maniatis Laboratory Manual, Post processing was performed using PIERCE SuperSignal staining kit.

CG47, CG-30, CG-23, and CG-35 are all hyperplastic prostate tissue sample. CG-37 is a normal prostate sample. The results indicate that the original PSA is expressed in hyperplastic, but not normal, prostate tissue (right) while the PSA variant (denoted PSALM) is weakly present hyperplastic prostate and highly expressed in normal tissue. Neither molecule is present in detectable levels in the spleen control.

C. Therapeutic Uses and Compositions Utilizing the PSA Variant Product

The PSA variant product of the invention is generally useful in treating diseases and disorders which are characterized by a lower than normal level of PSA variant expression, and or diseases which can be cured or ameliorated by raising the level of the PSA variant product, even if the level is normal.

PSA variant products or fragments may be administered by any of a number of routes and methods designed to provide a consistent and predictable concentration of compound at the target organ or tissue. The product-containing compositions may be administered alone or in combination with other agents, such as stabilizing compounds, and/or in combination with other pharmaceutical agents such as drugs or hormones.

PSA variant product-containing compositions may be administered by a number of routes including, but not limited to oral, intravenous, intramuscular, transdermal, subcutaneous, topical, sublingual, or rectal means as well as by nasal application. PSA variant product-containing compositions may also be administered via liposomes. Such administration routes and appropriate formulations are generally known to those of skill in the art.

The product can be given via intravenous or intraperitoneal injection. Similarly, the product may be injected to other localized regions of the body. The product may also be administered via nasal insufflation. Enteral administration is also possible. For such administration, the product should be formulated into an appropriate capsule or elixir for oral administration, or into a suppository for rectal administration.

The foregoing exemplary administration modes will likely require that the product be formulated into an appropriate carrier, including ointments, gels, suppositories. Appropriate formulations are well known to persons skilled in the art.

Dosage of the product will vary, depending upon the potency and therapeutic index of the particular polypeptide selected.

A therapeutic composition for use in the treatment method can include the product in a sterile injectable solution, the polypeptide in an oral delivery vehicle, the product in an aerosol suitable for nasal administration, or the product in a nebulized form, all prepared according to well known methods. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof.

### EXAMPLE III

Screening Methods for Activators and Deactivators

The present invention also includes an assay for identifying molecules, such as synthetic drugs, antibodies, peptides, or other molecules, which have a modulating effect on the activity of the PSA variant product, e.g. activators or deactivators of the PSA variant product of the present invention. Such an assay comprises the steps of providing an PSA variant product encoded by the nucleic acid sequences of the present invention, contacting the PSA variant protein with one or more candidate molecules to determine the candidate molecules modulating effect on the activity of the PSA variant product, and selecting from the molecules a candidate's molecule capable of modulating PSA variant product physiological activity.

PSA variant product, its catalytic or immunogenic fragments or oligopeptides thereof, can be used for screening therapeutic compounds in any of a variety of drug screening techniques. The fragment employed in such a test may be free in solution, affixed to a solid support, borne on a cell membrane or located intracellularly. The formation of binding complexes, between PSA variant product and the agent being tested, may be measured. Alternatively, the activator or deactivator may work by serving as agonist or antagonist, respectively, of the PSA variant receptor and their effect may be determined in connection with the receptor.

Another technique for drug screening which may be used provides for high throughput screening of compounds having suitable binding affinity to the PSA variant product is described in detail by Geysen in PCT Application WO 84/03564, published on Sep. 13, 1984. In summary, large numbers of different small peptide test compounds are synthesized on a solid substrate, such as plastic pins or some other surface. The peptide test compounds are reacted with the full PSA variant product or with fragments of PSA variant product and washed. Bound PSA variant product is then detected by methods well known in the art. Substantially purified PSA variant product can also be coated directly onto plates for use in the aforementioned drug screening techniques. Alternatively, non-neutralizing antibodies can be used to capture the peptide and immobilize it on a solid support.

Antibodies to the PSA variant product, as described in Example IV below, may also be used in screening assays according to methods well known in the art. For example, a “sandwich” assay may be performed, in which an anti-PSA variant antibody is affixed to a solid surface such as a microtiter plate and PSA variant product is added. Such an assay can be used to capture compounds which bind to the PSA variant product. Alternatively, such an assay may be used to measure the ability of compounds to influence with the binding of PSA variant product to the PSA variant receptor, and then select those compounds which effect the binding.

### EXAMPLE IV

Anti-PSA Variant Antibodies

A. Synthesis

In still another aspect of the invention, the purified PSA variant product is used to produce anti-PSA variant antibodies which have diagnostic and therapeutic uses related to the activity, distribution, and expression of the PSA variant product, in particular diagnostic application in identification of prostate cancer, (distinguishing between malignant and benign states) and as targeting means for delivery of cytotoxic compounds to tumor cells.

Antibodies to PSA variant product may be generated by methods well known in the art. Such antibodies may include, but are not limited to, polyclonal, monoclonal, chimeric, humanized, single chain, Fab fragments and fragments produced by an Fab expression library. Antibodies, i.e., those which inhibit dimer formation, are especially preferred for therapeutic use.

PSA variant product for antibody induction does not require biological activity; however, the protein fragment or oligopeptide must be antigenic. Peptides used to induce specific antibodies may have an amino acid sequence consisting of at least five amino acids, preferably at least 10 amino acids of the sequences specified in any of the SEQ ID NO: 7 to 12 Preferably they should mimic a portion of the amino acid sequence of the natural protein and may contain the entire amino acid sequence of a small, naturally occurring molecule. The antibodies may also distinguish antibodies, i.e. antibodies which bind to an amino acid sequence present in the PSA variant and not in the original PSA sequence. For the production of said distinguishing antibodies “distinguishing amino acid sequences” may be used for example having the sequence CQAELSPPTQHPSPDREL, derived from amino acids between the positions 33-51 in the protein sequence of invention as depicted in SEQ ID NO: 7, which is depicted in SEQ ID NO: 16. Another example of polypeptide sequence that may be used for the production of said distinguishing antibodies “distinguishing amino acid sequences” is polypeptide depicted in SEQ ID NO: 21, having the sequence PSQIPAPSCFTKEQVPRHLC, corresponding to amino acid residues 85-104 of the variant encoded by SEQ ID NO: 6.

Antibodies may also be produced by inducing in vivo production in the lymphocyte population or by screening recombinant immunoglobulin libraries or panels of highly specific binding reagents as disclosed in Orlandi et al. (Proc. Natl. Acad. Sci. 86:3833-3837, 1989)), and Winter G and Milstein C., (Nature 349:293-299, (1991)).

Antibody fragments which contain specific binding sites for PSA variant protein may also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse W. D. et a., Science 256:1275-1281, (1989)).

Production of Antibodies

Human and mouse cDNA fragments were subcloned into the pET-28(a-c) vectors (Novagen,USA). DNA was prepared from positive clones and introduced into the E.coli strain DE3 according to the manufacturer's recommendations. After induction, extracts were electrophoresed through a 10% SDS-PAGE. Fxtracts were prepared from clones that expressed the expected size protein and loaded on a nickel-agarose column. The His containing proteins were isolated from the column according to the manufacturer's recommendations and used in injections. Polyclonal antibodies against human PSAL peptide were prepared by immunizing rabbits with 3-4 injections of 0.5 mg of the purified protein at 1-2 week intervals. Animals were bled 10 days after the final booster. Serum was separated from the blood and stored at −80C. The peptide defined above as “distinguishing amino acid sequence” was used for immunization.

D. Diagnostic Applications of Antibodies

A variety of protocols for competitive binding or immunoradiometric assays using either polyclonal or monoclonal antibodies with established specificities are well known in the art. Such immunoassays typically involve the formation of complexes between PSA variant product and its specific antibody and the measurement of complex formation. A two-site, monoclonal-based immunoassay utilizing monoclonal antibodies reactive to two non-interfering epitopes on a specific PSA variant product is preferred, but a competitive binding assay may also be employed. These assays are described in Maddox D. E., et al., (J. Exp. Med. 158:1211, (1983)).

Antibodies which specifically bind PSA variant product are useful for the diagnosis of conditions or diseases characterized by expression of PSA variant protein, in particular prostate cancer. Alternatively, such antibodies may be used in assays to monitor patients being treated with PSA variant product, its activators, or its deactivators. Diagnostic assays for PSA variant protein include methods utilizing the antibody and a label to detect PSA variant product in human body fluids or extracts of cells or tissues. The products and antibodies of the present invention may be used with or without modification. Frequently, the proteins and antibodies will be labeled by joining them, either covalently or noncovalently, with a reporter molecule. A wide variety of reporter molecules are known in the art.

A variety of protocols for measuring PSA variant product, using either polyclonal or monoclonal antibodies specific for the respective protein are known in the art. Examples include enzyme-linked immunosorbent assay (ELISA), radioimmunoassay (RIA), and fluorescent activated cell sorting (FACS). As noted above, a two-site, monoclonal-based immunoassay utilizing monoclonal antibodies reactive to two non-interfering epitopes on PSA variant product is preferred, but a competitive binding assay may be employed. These assays are described, among other places, in Maddox, et al. (supra). Such protocols provide a basis for diagnosing altered or abnormal levels of PSA variant product expression. Normal or standard values for PSA variant product expression are established by combining body fluids or cell extracts taken from normal subjects, preferably human, with antibody to PSA variant product under conditions suitable for complex formation which are well known in the art. The amount of standard complex formation may be quantified by various methods, preferably by photometric methods. Then, standard values obtained from normal samples may be compared with values obtained from samples from subjects potentially affected by disease. Deviation between standard and subject values establishes the presence of disease state.

The antibody assays are useful to determine the level of PSA variant present in a body fluid sample, or in a particular tissue, e.g., biopsied tumor tissue, for example from the prostate gland, as an indication of whether PSA variant is being overexpressed or underexpressed in the tissue, or as an indication of how PSA variant levels are responding to drug treatment.

C. Immunohistochemical Staining:

Human prostate micron sections were prepared using a R. Gung microtome and fixed on slides pretreated with 2% Tespa (Sigma, USA). Deparaffinization was performed for 30 minutes at 80° C. Hydration was executed by immersing the slides twice in xylene (5 minutes each), twice in 100% ethanol (5 minutes each), twice in 95% ethanol (5 minutes each),once in 70% ethanol (5 minutes), and once in PBS pH7.4 (10 minutes). After incubation in 50 μl/slide of 1.5 mg/ml hyaluronidase in PBS pH6.5 for 1 hour at 37° C. the slides were washed in PBS pH 7.4 (10 minutes). 50 μl/slide of 0.3% H₂O₂ in PBS pH 7.4 were added for 10 minutes after which they were washed in PBS pH 7.4 (10 minutes).

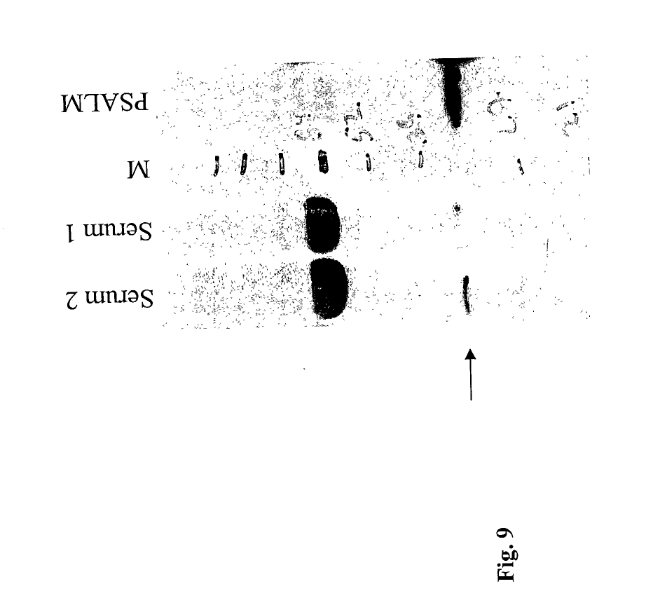

Blocking was performed by addition of 50 μl/slide of normal goat serum containing 20% trasylol at 37° C. for 10 minutes. Rabbit anti-testilin antibodies were diluted 1:50in 10% blocking solution (normal goat serum containing 20% trasylol (Bayer, Germany) and interacted with the slides for 18 hours at 4° C. Then the slides were washed 3 times in PBS pH 7.4 and immersed for 10 minutes in PBS pH 7.4. Horse raddish peroxidase (HRP) conjugated goatanti rabbit antibodies (Sigma, USA) in PBS pH7.4 containing 20% trasylol (Bayer, Germany) diluted 1:40, were added for 30 minutes at room temperature, in the dark, following by wash in PBS pH7.4 for 10 minutes. For HRP reaction 0.4 mg/ml of the substrate (3′3′ diaminbenzoidin) was added in the dark for 10 minutes. Following 3 washes in PBS pH 7.4 and immersion for 10 minutes in PBS pH 7.4, staining of the slides was performed with 1% methylene blue in PBS pH 7.4 for 5 min. Following two washes in water, dehydration was carried out by immersing the slides 3 times in 70% ethanol, 3 times in 95% ethanol, 6 times in 100% ethanol and 6 times in xylene. Mounting was performed with MERCOGLASS (Merk, USA).The results are shown in FIG. 9. The result indicate a high presence level of the PSA variant protein in the lumen-lining of prostate epithelial cells (color gold, left picture, vs. the pre-immune serum control on the right picture, where no gold color is detectable.)

D. Therapeutic Uses of Antibodies

In addition to their diagnostic use the antibodies may have a therapeutical utility in blocking or decreasing the activity of the PSA variant product in pathological conditions where its activity or concentration are too high, for example in prostate cancer. In addition, the antibodies may be conjugated to cytotoxic compounds and thus may serve as means for targeting the cytotoxic moiety only to cancer cells which express membrane-bond PSA variant product.

The antibody employed is preferably a humanized monoclonal antibody, or a human Mab produced by known globulin-gene library methods. The antibody is administered typically as a sterile solution by IV injection, although other parenteral routes may be suitable. Typically, the antibody is administered in an amount between about 1-15 mg/kg body weight of the subject. Treatment is continued, e.g., with dosing every 1-7 days, until a therapeutic improvement is seen.

### EXAMPLE V

Immuno-histochemistry

Immunohistochemical staining was performed using HISTOSTAIN SP kit (Zymed Laboratories INC.).

Human prostate micron sections were prepared using a R. Gung microtome and fixed on superfrost slides with 2% Tespa. Deparaffinization was performed for 10 mins. at xylen. Hydration three times 100% ethanol and once 95% ethanol. The slides were washed in Ddw, following incubation with 3% H₂O₂ for 5 mins. After incubation the slides were washed twice in ddw, and twice in 0.05M Tris Hcl Ph 7.6 (optimax wash buffer, BioGenex).

Blocking was performed with serum blocking solution (ready to use, reagent A, Zymed) 100 ul each slide incubate 10 mins. Primary antibody was diluted 1:50 in antibody diluent reagent solution (Zymed), and incubated in moist chamber with the slides for 1 hour.

Following washing (three times in optimax buffer) the slides were incubated with 100 μl biotinylated second antibody ready to use (reagent 13 Zymed), for 10 mins, then washed three more times in optimax buffer. The slides were incubated with 100 μl enzyme conjugate HRP-streptavidin ready to use (reagent C, Zymed) for 10 mins, and washed twice in optimax buffer. Then 100 μl substrate (liquid DAB substrate, Zymed) were added for 3 mins.

Following the incubation with the substrate, the slides were washed twice in ddw and stained with Hematoxylen solution (Zymed) for 2 mins. Then the slides were washed in tap water for 1 hour. The dehydration was carried out by immersing the slides 2 times in 95% ethanol, 3 times in 100% ethanol, 3 times in Zylen. Mounting was performed with mounting solution (Zymed).

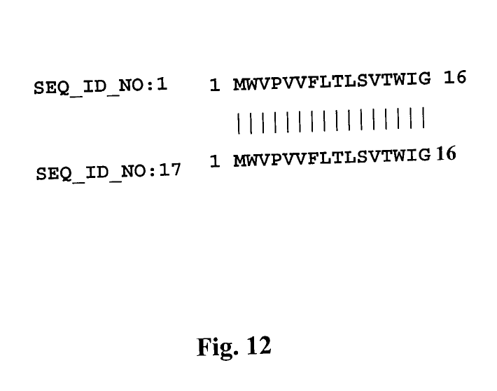

The results are shown in FIG. 12. The results indicate a high presence level of the protein (derived by alternative splicing from the KLK-2 gene) in the lumen-lining of prostate epithelial cells (gold color, left picture, vs. the pre-immune serum control on the right picture, where no gold color is detectable).

### EXAMPLE VI

Hormonal Regulation of PSALM mRNA Expression

The androgenic transcriptional activation of the KLK2 and KLK3 genes has been studied extensively (e.g. Young, C. Y., 1991, Cancer Research, 51, 3748-3752). The effects of androgenic stimulation on the expression of PSA-LM were analyzed using the androgen-responsive human prostatic adenocarcinoma cell line LNCaP (ATCC, Manassas, Va.). LNCaP cell line was propagated in RPMI 1640 containing 10% fetal calf serum. When cells reached 70% confluency, the calture medium was replaced by phenol red-free RPMI 1640 supplemented with 2% charcoal-stripped fetal calf serum for 24 h, followed by addition of 10 nM methyltrienolone (R1881, PerkinElmer Life Sciences) or testosterone (Fluka) for periods of 9-48 h,

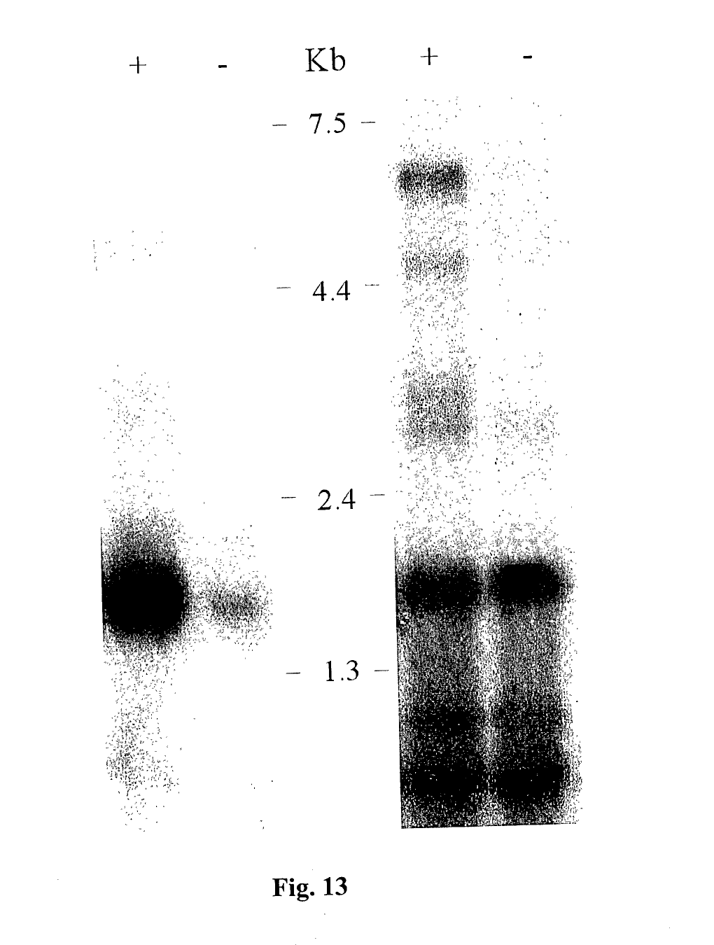

After 24 h of hormonal depletion, cells were fed on charcoal-striped serum in the absence or presence of either testosterone or a synthetic androgen (R1881). Northern blot analysis using of a probe specific to the original PSA, as depicted in SEQ ID NO: 17, showed a strong induction of the 1.6-kb PSA-encoding transcript after exposure to 10 nM R1881 (FIG. 13, left panel) or testosterone (data not shown) for 9 h, conditions previously shown to yield maximal expression of PSA mRNA (Young, C. Y., 1991, Cancer Research, 51, 3748-3752). Under these conditions, a 2-6-fold up-regulation of the 3-, 5-, and 6.5-kb transcripts was also detected using a 780-bp probe derived from intron 1 that spans the PSA-LM open reading frame (ORF) without the signal peptide common to PSA, depicted in SEQ ID NO: 22 (FIG. 13, right panel). This probe, depicted in SEQ ID NO: 22, specifically identifies the new splice variants of PSALM, depicted in SEQ ID NOs: 1-5. Higher levels of PSA and PSA-LM mRNAs were also observed up to 48 h (data not shown).

### EXAMPLE VII

PSALM and KLM Protein Expression and Secretion.

Recombinant Protein Expression and Secretion

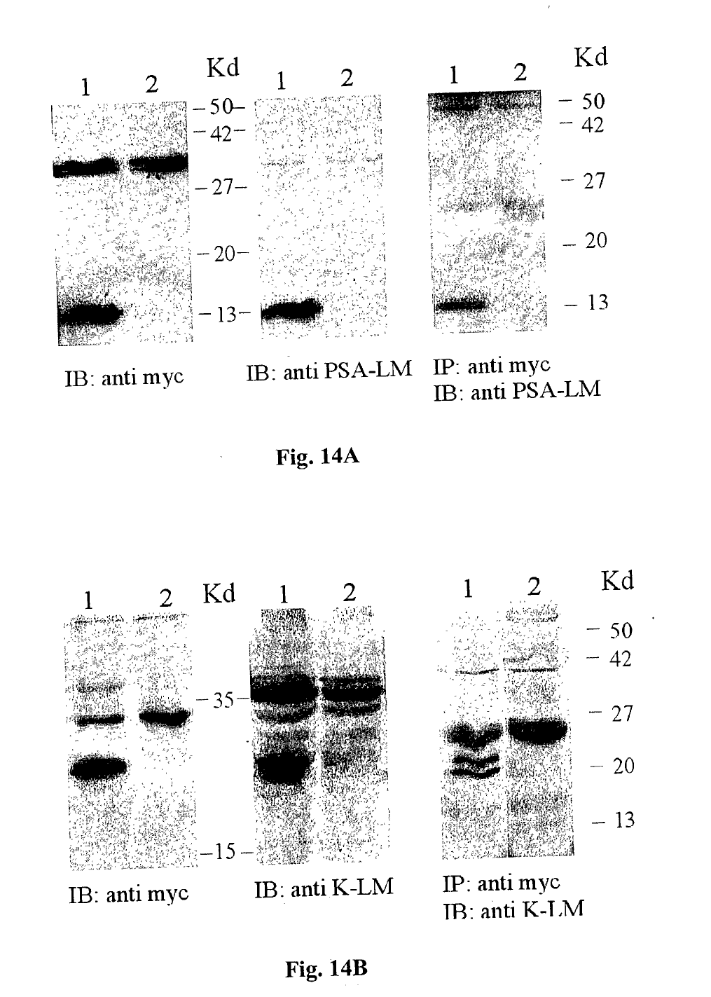

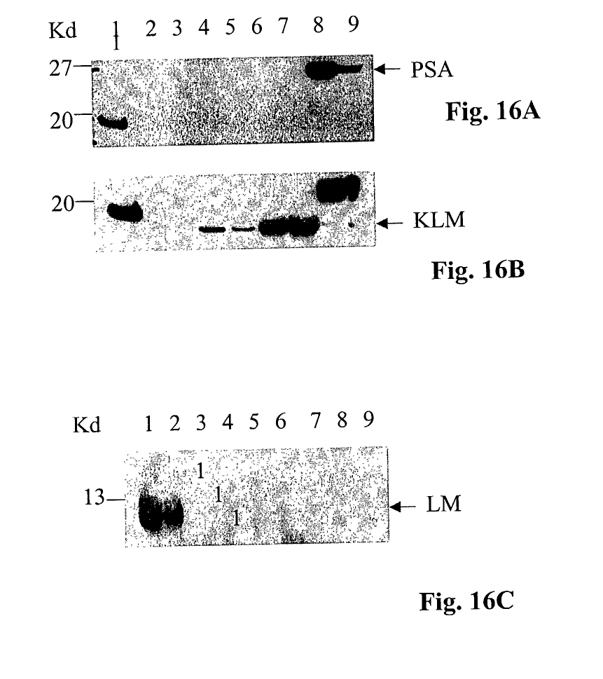

The complete coding sequences of K-LM (encoding protein depicted in SEQ ID NO: 12) or PSA-LM (encoding protein depicted in SEQ ID NO: 1) were amplified by RT-PCR and cloned into the pcDNA4/Myc-His mammalian expression vector (Invitrogen), enabling the synthesis of proteins fused to the Myc and His epitope tags at the C terminus. LNCaP cells, at about 70% confluency, were transfected with these expression constructs using LipofectAMINE (Invitrogen), according to the manufacturer's instructions. As a negative control, LNCap cells transfected with pcDNA4 vector alone were used (FIGS. 16A and B, lanes 2). After 48 h, spent media were collected, and cells were washed with phosphate-buffered saline, harvested, and lysed in 50 mM Tris, pH 7.5, 150 mM NaCl, 1% Triton X-100, containing a protease inhibitor mixture (Complete™, Roche Molecular Biochemicals). Proteins from the culture medium were immunoprecipitated using c-Myc-conjugated agarose beads (9E10 AC, Santa Cruz Biotechnology Inc., Santa Cruz, Calif.), or specific polyclonal antibodies, raised against synthetic peptides derived from the amino acid sequences of PSALM and KLM, depicted in SEQ ID NOs: 16 and 21, respectively). Cell lysates (60 μg) were fractionated by 15% SDS-PAGE and subjected to immunodetection with a monoclonal anti-Myc antibody (FIGS. 14A and B, left panels), followed by analysis with rabbit sera immunized against PSALM (FIG. 14A middle panel) or against K-LM (FIG. 14B middle panel).

The immunoblot analysis of the transfected cell lysates showed expression of PSA-LM and K-LM (FIGS. 14A and B, left and middle panels). The observed sizes of these recombinant proteins (13 and 20 kDa, respectively) are similar to those predicted from their amino acid sequence (11 and 15 kDa), with the addition of about 3 kDa of the epitope tags. The additional bands observed in FIG. 14B most likely represent cross-reacting proteins, as they were not detected with other sera raised against K-LM (not shown).

The secretion of these proteins to the growth medium of the transfected cell cultures was confirmed by immunoprecipitation using anti- Myc-coupled beads, followed by immunodetection with the specific antibodies (FIGS. 14, A and B, right panels). Equal loading is shown by the intensity of the nonspecific bands.

Secretion to the Seminal Fluid and Expression in BPH (Benign Prostatic Hyperplasia)

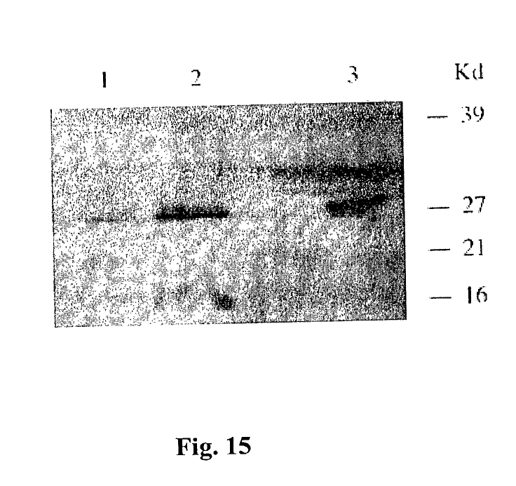

Seminal plasma, obtained from ejaculates after removal of spermatozoa by centrifugation, was diluted in phosphate-buffered saline containing a protease inhibitor mixture. Proteins from seminal plasma (25 μg, FIG. 15 lanes 1 and 2) and a BPH sample (100 μg, FIG. 15, lane 3) were separated by SDS-PAGE on 15% gels, transferred to polyvinylidene difluoride membranes, followed by immunodetection with K-LM-specific polyclonal antibodies (raised against polypeptide depicted in SEQ ID NO: 21). Immunoblot analysis of samples of semen plasma and BPH identified a major form of the native K-LM protein, appearing as 27 kDa (FIG. 15). The appearance of this and fainter bands in these samples, which are bigger than the expected size of this protein, could be due to post-translational modifications, such as glycosylation. This assumption gains support from the presence of several putative glycosylation sites in its amino acid sequence (not shown). These results demonstrate that native KLM variant protein, depicted in SEQ ID NO: 12, is secreted to seminal fluid and expressed in BPH.

### EXAMPLE VIII

PSALM and KLM Protein Expression in Lung.

For the western blot analysis of endogenous expression of the original PSA (SEQ ID NO: 18), KLM (SEQ ID NO: 12) and PSA-LM (SEQ ID NO: 7), cells and tissues protein extracts were separated by 12% SDS-PAGE and subjected to immunoreaction with K-LM specific polyclonal antibody (FIG. 16B), followed by analysis with a monoclonal anti PSA antibody (FIG. 16A). Alternatively, tissues protein extracts were separated by 12% SDS-PAGE and subjected to immunoreaction with a monoclonal antibodies anti PSA-LM (FIG. 16C). The tissue samples examined were as follows: in FIGS. 16A and B: recombinant KLM and PSA-LM expressed in bacteria (lanes 1 and 2, respectively), H1299—non-small cell lung cancer cell line (lane 4), LNCap-prostate carcinoma cell line (lane 5), CG-314-Lung bronchioloaveolar carcinoma obtained from patients (lane 6), CG-314N—Normal lung sample, obtained from patients (lane 7), CG-225—Benign prostatic hyperplasia sample, obtained from lo patients (lane 8), CG-259—Benign prostatic hyperplasia sample, obtained from patients (lane 9). In FIG. 16C, the tissue samples examined were as follows: CG-314N—Normal lung sample, obtained from patients (lane 1), CG-314—Lung bronchioloaveolar carcinoma sample, obtained from patients (lane 2)) CG-312N—Normal breast (lane3) sample, obtained from patients, CG-312—breast multifocal infiltrating ductal carcinoma sample, obtained from patients (lane 4), CG-255-6—Prostate adenomatous and stromal hyperplasia sample, obtained from patients (lane 5), CG-261-8—Prostate within normal limits sample, obtained from patients (lane 6), CG-243-4—Prostate hypertrophy with focal squamous metaplsia sample, obtained from patients (lane 7), CG-225—Benign prostatic hyperplasia sample, obtained from patients (lane 8), CG-259—Benign prostatic hyperplasia sample, obtained from patients (lane 9).

As demonstrated in FIG. 16B, the observed size of the recombinant K-LM protein (lane 1) is similar to the predicted size based on amino acid sequence, with the addition of about 3 Kd of the epitop tags. Endogenous K-LM protein could be detected in cancer and normal lung tissue (lanes 6 and 7, respectively FIG. 16B), and also in H1299 cell line of lung cancer. Interestingly, a bigger band of the expected size of K-LM protein, appear in the two benign prostatic hyperplasia (BPH) samples that were analyzed (lanes 8 and 9). The appearance of this band could be due to post translational modifications. The original PSA protein (FIG. 16A) could be detected only in the two BPH samples (lanes 8 and 9, upper panel) and not in the lung samples (lanes 6 and 7). As is demonstrated in FIG. 16C, the observed size of the PSA-LM protein corresponds to the size that is predicted from the amino acid sequence of PSA-LM, and is about 11 Kd. Endogenous PSA-LM protein could be detected in normal and cancer lung tissues (lane 1 and 2, respectively). Endogenous PSA-LM could not be detected in one normal and one cancer breast tissues (lane 3 and 4, respectively), and in four different prostate tissues (lanes 5-9) that were analyzed.

To further demonstrate PSA-LM expression in the epithelial cells of lung, an immunohistochemistry of Lung sections using, anti PSA-LM monoclonal antibody was conducted.

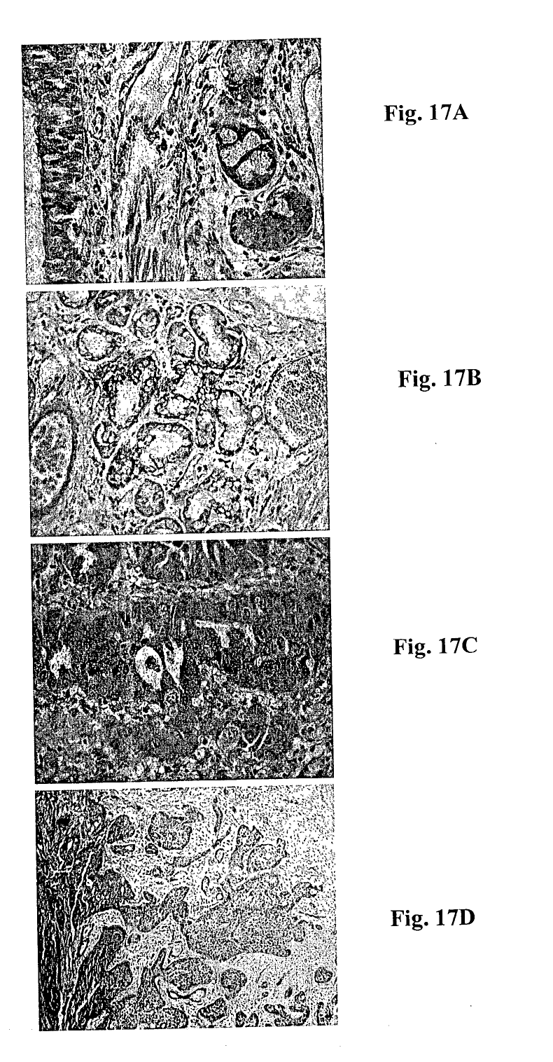

FIG. 17 demonstrates cytoplasmic staining within the epithelial cells of the bronchus wall (FIG. 17A), the epithelial cells lining the submucosal glands (FIGS. 17A and B), moderately differentiated adenocarcinoma (FIG. 17C) and squamous cell carcinoma (FIG. 17D). All the slides were counterstained with hematoxylin.

Immunohistochemistry analysis of lung sections using PSA-LM monoclonal antibody showed specific staining in normal epithelium (FIGS. 17A and B) and neoplastic epithelial cells (FIGS. 17C and D). The immunohistochemical staining was performed by Patho-lab LTD (http://www.patholab.com).

Although the invention has been described with reference to specific methods and embodiments, it is appreciated that various modifications and changes may be made without departing from the invention.

1. An isolated nucleic acid sequence selected from:
(i) the nucleic acid sequence set forth in any one of SEQ ID NO: 1 to SEQ ID NO: 6;
(ii) nucleic acid sequence having at least 90% identity with the entire length of the sequence of (a) and
(iii) fragments of (a) or (b) of at least 20 consecutive nucleotides provided that the fragment contains a sequence which is not present in the original sequence of PSA from which the sequences of (a) have been varied by alternative splicing.

2. An isolated nucleic acid sequence complementary to the nucleic acid sequence of claim 1.

3. An amino acid sequence selected from:
(i) an amino acid sequence encoded by the isolated nucleic acid sequence of claim 1;
(ii) fragments of the amino acid sequence of (a) having at least 10 consecutive amino acids, provided that said fragment contains a sequence which is not present in the original sequence of PSA from which the amino acid sequence of (a) have been varied by alternative splicing;
(iii) analogs of the amino acid sequences of (a) or (b) in which one or more amino acids has been added, deleted, replaced, or chemically modified without substantially altering the biological activity of amino acid sequences of (a) and (b).

4. An amino acid sequence according to claim 3, as set forth in any one of SEQ ID NO: 7 to SEQ ID NO: 12.

5. An isolated nucleic acid sequence encoding the amino acid sequence of claim 3.

6. An isolated nucleic acid sequence coding for an isolated amino acid sequence of SEQ ID NO: 7 to SEQ ID NO: 12.

7. An expression vector comprising the nucleic acid sequences of claim 1 and control elements for the expression of the nucleic acid sequence in a suitable host cell.

8. An expression vector comprising the nucleic acid sequence of claim 2, and control elements for the expression of the nucleic acid sequence in a suitable host cell.

9. A host cell transfected by the expression vector of claim 7.

10. A host cell transfected by the expression vector of claim 8.

11. A purified antibody that binds specifically to an amino acid sequence encoded by the isolated nucleic acid sequence of claim 1.

12. A purified antibody that binds specifically to an amino acid sequence present in any one of the amino acid sequence of claim 3, and which is not present in the original PSA sequence.

13. A purified antibody that binds specifically to an amino acid sequence present in any one of the amino acid sequence of claim 4, and which is not present in the original PSA sequence.

14. The antibody of claim 12, wherein the antibody is a monoclonal antibody.

15. The antibody of claim 13, wherein the antibody is a monoclonal antibody.

16. A purified antibody fragment comprising an antigen-binding domain of an anti-PSA variant product antibody of claim 12.

17. A purified antibody fragment comprising an antigen-binding domain of an anti-PSA variant product antibody of claim 13.

18. A purified antibody according to claim 12 conjugated to a cytotoxic or cytostatic compound.

19. A pharmaceutical composition comprising a pharmaceutically acceptable carrier and, as an active ingredient, an agent selected from:
(i) an expression vector comprising a nucleic acid sequence of SEQ ED NO: 1 to SEQ ID NO: 6, a nucleic acid having at least 90% identity with the entire length thereof and fragmendts thereof having at least 20 consecutive nucleotides provided that the fragment contain a sequence which is not present in the original sequences of PSA from which the said sequence have been varied;
(ii) the amino acid sequence of claim 3; and
(iii) an amino acid sequence as set forth in any one of SEQ ID NO: 7 to SEQ ID NO: 12.

20. A pharmaceutical composition comprising a pharmaceutically acceptable carrier and, as an active ingredient, an agent selected from:
(b) an expression vector of claim 8; and
(c) the purified antibody of any one of claim 11.

21. A method for detecting a PSA variant in a biological sample, the method comprising:
(i) isolating nucleic acid material from the biological sample;
(ii) contacting a nucleic acid sequence of claim 1 to the nucleic acid material from the biological sample under conditions that enable hybridization; and
(iii) detecting hybridization complexes;
wherein the presence of hybridization complexes indicates the presence of a PSA variant nucleic acid in the biological sample.

22. A method for detecting a PSA variant in a biological sample, the method comprising:
(i) isolating nucleic acid material from the biological sample;
(ii) contacting a nucleic acid sequence of claim 2 to the nucleic acid material from the biological sample under conditions that enable hybridization; and
(iii) detecting hybridization complexes;
wherein the presence of hybridization complexes indicates the presence of a PSA variant nucleic acid in the biological sample.

23. The method of claim 21, wherein the biological sample is from an individual, and the presence of a PSA variant in the biological sample indicates the presence of or predisposition to prostate cancer in the individual.

24. The method of claim 22, wherein the biological sample is from an individual, and the presence of a PSA variant in the biological sample indicates the presence of or predisposition to prostate cancer in the individual.

25. The method of claim 23, wherein the presence of a PSA variant in the biological sample indicates the presence of or predisposition to prostate cancer in the tissue.

26. The method of claim 24, wherein the presence of a PSA variant in the biological sample indicates the presence of or predisposition to prostate cancer in the tissue.

27. The method of claim 21, wherein the biological sample is from an individual, and the presence of a PSA variant in the biological sample indicates the presence of the malignancy of the cancer or the stage of the cancer.

28. The method of claim 21, wherein the biological sample is from an individual, and the presence of a PSA variant in the biological sample indicates the presence of the malignancy of the cancer or the stage of the cancer.

29. The method of claim 21, wherein the tissue is ovary, breast, lung or salivary gland tissue.

30. The method of claim 22, wherein the tissue is ovary, breast, lung or salivary gland tissue.

31. A method for determining the level of nucleic acid sequences of PSA variants in a biological sample comprising:
(i) hybridizing to nucleic acid material of the biological sample any one of the nucleic acid sequences of claim 1; and
(ii) determining the amount of hybridization complexes and normalizing the amount to provide the level of the PSA variant nucleic acid sequences in the sample.

32. A method for determining the level of nucleic acid sequences of PSA variants in a biological sample comprising:
(i) hybridizing to nucleic acid material of the biological sample any one of the nucleic acid sequences of claim 2; and
(ii) determining the amount of hybridization complexes and normalizing the amount to provide the level of the PSA variant nucleic acid sequences in the sample.

33. A method for determining the ratio between the level of a PSA variant in a first biological sample and the level of the original PSA sequence, in a second biological sample, the method comprising:
(i) determining the level of the PSA variant in the first biological sample according to the method of claim 31;
(ii) determining the level of the PSA original sequence in the second biological sample; and
(iii) comparing the levels obtained in (one) and (two) to obtain a ratio.

34. A method for determining the ratio between the level of a PSA variant in a first biological sample and the level of the original PSA sequence, in a second biological sample, the method comprising:
(i) determining the level of the PSA variant in the first biological sample according to the method of claim 32;
(ii) determining the level of the PSA original sequence in the second biological sample; and
(iii) comparing the levels obtained in (one) and (two) to obtain a ratio.

35. A method according to claim 3 where the nucleic acid sequence is present in a nucleic acid chip.

36. A method according to claim 35, wherein the method is for detection of the presence of prostate cancer, detection of predisposition to prostate cancer, or evaluation of the malignancy of prostate cancer.

37. A method for identifying a candidate compound capable of binding to a PSA variant product and modulating its activity, the method comprising:
(i) providing a polypeptide comprising an amino acid sequence as set forth in any one of SEQ ID NOS: 7 to 12, or a fragment of such a sequence, having at least 10 consecutive amino acids;
(ii) contacting a candidate compound with the amino acid sequence; and
(iii) determining the effect of the candidate compound on the biological activity of the polypeptide and selecting those candidate compounds that show a significant effect on the biological activity.

38. A method according to claim 37, wherein the compound is an activator and the measured effect is an increase in the biological activity.

39. A method according to claim 37, wherein the compound is a deactivator and the effect is a decrease in the biological activity.

40. An activator of the amino acid sequence of claim 3.

41. A deactivator of the amino acid sequence of claim 3.

42. A method for detecting a PSA variant product in a biological sample, the method comprising:
(i) contacting the biological sample with the antibody of claim 11, thereby forming an antibody-antigen complex; and
(ii) detecting the antibody-antigen complex
wherein the presence of the antibody-antigen complex indicates the presence of a PSA variant product in the biological sample.

43. A method for determining the level of amino acid sequences of PSA variants of claim 3 in a biological sample, the method comprising:
(i) contacting the biological sample with the antibody of claim 11, thereby forming an antibody-antigen complex; and
(ii) detecting an amount of the antibody-antigen complex and normalizing the amount to provide the level of the amino acid sequence in the sample.

44. A method for determining the ratio between the level of a PSA variant having the amino acid sequences of claim 3 in a first biological sample and the level of the original PSA sequence from which the variant has been varied by alternative splicing, in a second biological sample, the method comprising:
(i) determining the level of the PSA variant amino acid sequence in the first biological sample according to the method of claim 43;
(ii) determining the level of the PSA original sequence in the second biological sample in the same manner; and
(iii) comparing the levels obtained in (a) and (b) to obtain the ratio.

45. A method according to claim 44, wherein the first and second biological samples are the same sample.

46. A method according claim 42, wherein the method is used for detecting the presence of prostate cancer or detecting pre-disposition to prostate cancer, or for detection of the malignancy of prostate cancer.

47. A method of detecting differential expression of a PSA variant nucleic acid in various tissues from an individual, the method comprising
(i) obtaining a sample from two or more tissues within an individual;
(ii) isolating nucleic acid material from the samples;
(iii) contacting a nucleic acid sequence of claim 1 to the nucleic acid material from the samples under conditions that enable hybridization; and
(iv) detecting hybridization complexes;
wherein the presence of hybridization complexes in one sample and not another sample indicates differential expression of the PSA variant nucleic acid.

48. A method of detecting differential expression of a PSA variant nucleic acid in various tissues from an individual, the method comprising
(i) obtaining a sample from two or more tissues within an individual;
(ii) isolating nucleic acid material from the samples;
(iii) contacting a nucleic acid sequence of claim 2 to the nucleic acid material from the samples under conditions that enable hybridization; and
(iv) detecting hybridization complexes;
wherein the presence of hybridization complexes in one sample and not in the other sample indicates differential expression of the PSA variant nucleic acid.

49. A method of eliciting an immune response in a mammal, the method comprising administering to the mammal an amount of a PSA variant product effective to generate antibodies or cause proliferation of PSA variant product-specific immune cells within the mammal.

50. The method of claim 49, wherein the PSA variant product is an amino acid sequence that distinguishes the PSA variant product from the original PSA product.

51. A method of targeting a compound to tumor cells expressing a PSA variant product in an individual, the method comprising:
(i) conjugating the compound to an antibody that binds specifically to a PSA variant product to form a conjugate;
(ii) administering to the individual an amount of the conjugate effective to deliver the compound to the tumor cells, thereby targeting the compound to the tumor cells expressing the PSA variant product.

52. The method of claim 51, wherein the antibody is a distinguishing antibody.

53. The method of claim 51, wherein the tumor cells are prostate tumor cells.

54. The method of claim 51, wherein the compound is a cytotoxic or cytostatic compound.

55. A method of expressing PSA variant polypeptides in vivo in an individual, the method comprising introducing into cells of the individual a PSA variant sequence that encodes a PSA product, thereby expressing a PSA variant polypeptide in vivo.

56. The method of claim 55, wherein the cells are introduced into cells of the individual by isolating the cells from the individual, introducing the PSA variant into the cells to produce engineered cells, and returning the engineered cells to the individual.

57. The method of claim 55, wherein the PSA variant is introduced into the cells using a viral plasmid vector.

58. The method of claim 55, wherein the PSA variant is operably linked to an inducible promoter.

59. The method of claim 55, wherein the inducible promoter is a radiation-inducible promoter.

60. The method of claim 55, wherein the cells are embryonic stem cells, hematopoietic stem cells, hepatocytes, fibroblasts, myoblasts, keratinocytes, endothelial cells, or bronchial epithelial cells.

61. The method of claim 55, wherein the PSA variant is introduced into the cells by administering to the individual a producer cell comprising the PSA variant in a retroviral vector, wherein the producer cell generates retroviral particles that encode a PSA variant product within the individual.
